(19) United States
(12) Patent Application Publication
(10) Pub. No.: US 2023/0065770 A1
(43) Pub. Date: Mar. 2, 2023

(54) DIALYSIS CONSUMABLES INVENTORY TRACKING

(71) Applicant: Medtronic, Inc., Minneapolis, MN (US)

(72) Inventors: Veena Bangalore Mahadevappa, Bangalore (IN); Arindam Ghosh Roy, Bangalore (IN)

(21) Appl. No.: 17/464,165

(22) Filed: Sep. 1, 2021

(51) Int. Cl. G16H 40/40; G16H 40/67; G16H 40/20; G16H 20/40

(52) U.S. Cl. CPC G16H 40/40; G16H 40/67; G16H 40/20; G16H 20/40

(57) ABSTRACT

In some examples, a dialysis system is configured to track consumables used by a dialysis machine to deliver a dialysis treatment to a patient in order to facilitate the delivery of additional consumables for use in future dialysis treatments. The dialysis machine can be configured to use a cartridge to replenish a dialysate and/or a recharger configured to recharge the cartridge. The dialysis system may track a consumables inventory (e.g., stored locally with the patient, such as at the patient's home) based on an amount of dialysate provided to a dialyzer over the course of the dialysis treatment and a dialysate prescription of the patient. The dialysis system may communicate the consumables inventory and/or a consumption of consumables associated with the patient to a remote device. The remote device may schedule a delivery of consumables to the patient based the consumables inventory and/or the consumption of consumables.

### TECHNICAL FIELD

This disclosure is related to dialysis.

### BACKGROUND

Dialysis systems may be used to remove waste products from blood of a patient when the kidneys of the patient are no longer able to adequately do so. During dialysis, a dialysis machine may generate or regenerate dialysate using specified concentrations of solute buffers, osmotic agents, cations, and/or other concentrates for biocompatibility with the patient. A dialysis machine may provide the dialysate to a dialyzer to cause the dialysate to remove the waste products from the blood of the patient. Some dialysis systems regenerate the spent dialysate using one or more sorbent cartridges and one or more dialysate chemicals before returning the regenerated dialysate to the dialyzer for continued removal of waste products over the course of the dialysis treatment.

### SUMMARY

This disclosure describes a dialysis system configured to track an inventory of consumables associated with a patient, where a dialysis machine and a recharger are configured to use the consumables during one or more dialysis treatments. The dialysis system includes a dialysis machine including a dialyzer configured to enable the transfer of the waste products from the blood of the patient to a dialysate. The dialysis system is configured to regenerate the spent dialysate using one or more cartridges (e.g., sorbent cartridges) and one or more dialysate chemicals before returning the regenerated dialysate to the dialyzer for continued removal of waste products over the course of the dialysis treatment. Following a dialysis treatment, the cartridge may be recharged for use in subsequent dialysis treatments using one or more recharge chemicals. The dialysis system is configured to track a quantity of consumables consumed during a dialysis treatment and/or the recharging based on a dialysate prescription and an amount of dialysate provided to the dialyzer over the course of the dialysis treatment.

In some examples, the dialysis system is configured to communicate with a remote device, such as a supply server, to assist the patient in maintaining a sufficient inventory of consumables for a prescribed treatment regimen. For example, the dialysis system may be configured to interact with the supply server to automatically order consumables for delivery to the patient.

In some cases, the dialysis system delivers the dialysis treatment at the patient's home (or other non-clinic setting) and the devices, systems, and techniques described herein may help a patient maintain a sufficient inventory of consumables. For example, the dialysis system may track an inventory of consumables (e.g., stored locally with the patient, such as at the patient's home) based on an amount of dialysate provided to a dialyzer over the course of the dialysis treatment and a dialysate prescription of the patient. The dialysis system may communicate the inventory of consumables and/or a consumption of consumables associated with the patient to a remote device, which may schedule a delivery of consumables to the patient based the consumables inventory and/or the consumption of consumables.

In some examples, a dialysis system comprises a dialysis machine configured to deliver a dialysis treatment to a patient by at least transferring a dialysate through a dialyzer, wherein the dialysis machine is configured to recycle the dialysate using a cartridge; a recharger configured to recharge the cartridge following the dialysis treatment; and processing circuitry configured to: determine a dialysate prescription indicative of a composition of the dialysate, determine a dialysate amount indicative of an amount of dialysate transferred through the dialyzer during the dialysis treatment, and determine, using the dialysate amount and the dialysate prescription, a quantity of materials consumed during at least one of the dialysis machine delivering the dialysis treatment or the recharger recharging the cartridge.

In some examples, a dialysis system comprises a dialysis machine configured to deliver a dialysis treatment to a patient by at least transferring a dialysate through a dialyzer, and wherein the dialysis machine is configured to recycle the dialysate using a cartridge; a recharger configured to recharge the cartridge following the dialysis treatment, wherein the dialysis machine, the recharger, or the dialysis machine and the recharger are located at a first location; and processing circuitry configured to: determine a dialysate prescription indicative of a composition of the dialysate, determine a dialysate amount indicative of an amount of dialysate transferred through the dialyzer during the dialysis treatment, determine a quantity of materials consumed during at least one the dialysis machine delivering the dialysis treatment or the recharger recharging the cartridge based on the dialysate amount and the dialysate prescription, track an inventory of consumables associated with the patient based on the quantity of materials consumed, compare the inventory of consumables with a threshold consumables inventory associated with the patient, wherein the threshold consumables inventory is based on the dialysate prescription, and schedule a delivery of one or more consumables to the first location from a second location geographically displaced from the first location based on the consumables inventory tracked.

In some examples, a method comprises determining, by processing circuitry, a dialysate prescription indicative of a composition of a dialysate, wherein a dialysis system includes a dialysis machine and a recharger, wherein the dialysis machine is configured to deliver a dialysis treatment to a patient by at least transferring the dialysate through a dialyzer and recycling the dialysate using a cartridge; determining, by the processing circuitry, a dialysate amount indicative of an amount of dialysate transferred through the dialyzer during the dialysis treatment, and wherein the recharger is configured to recharge the cartridge; and determining, by the processing circuitry, a quantity of materials consumed during at least one of the dialysis machine delivering the dialysis treatment or the recharger recharging the cartridge, wherein the system circuitry determines the quantity of material consumed based on the dialysate amount and the dialysate prescription.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example dialysis system configured to remove waste from blood of a patient.

FIG. 2 is a conceptual diagram illustrating a dialysis machine and a recharger of the example dialysis system of FIG. 1.

FIG. 3 is a is a block diagram of an example network that can include the dialysis system of FIGS. 1 and 2.

FIG. 4 is a flow diagram of an example technique of using the dialysis system of FIGS. 1-3.

### DETAILED DESCRIPTION

In some examples, a dialysis system is configured to track consumables used by a dialysis machine to deliver a dialysis treatment to a patient in order to facilitate the delivery of additional consumables for use in future dialysis treatments. This disclosure describes a dialysis system configured to provide a patient with a dialysis treatment to remove waste products from the blood of a patient. The dialysis system is configured to use a dialyzer to enable the transfer of the waste products to a dialysate and discharge the spent dialysate from the dialyzer to a dialysate circuit. The dialysate circuit may be configured to regenerate the spent dialysate using one or more cartridges (e.g., sorbent cartridges) and one or more dialysate chemicals. The dialysis system may be configured to return the regenerated dialysate to the dialyzer, such that the dialysate may be used in a cyclic manner for continued removal of waste products over the course of a dialysis treatment.

Following a dialysis treatment, the cartridge may be recharged for use in subsequent dialysis treatments using one or more recharge chemicals. In examples, following the dialysis treatment, a user (e.g., a patient or patient caretaker) detaches the cartridge from a dialysis machine defining the dialysate circuit and transfers the cartridge to a recharger, which recharges the cartridges in preparation for a subsequent dialysis treatment.

The dialysis system requires that a sufficient quantity of consumable items be available on-hand for a patient to receive the dialysis treatment. For example, the dialysate chemicals used to regenerate the dialysate during the dialysis treatment may be consumable items expended during the dialysis treatment. The recharge chemicals used to recharge the cartridge may be consumable items expended following the dialysis treatment. Further, the cartridge may have a recommended lifetime (e.g., a limited number of times it can be used for treatment and subsequently recharged). Thus, when a patient utilizes the dialysis system according to a prescribed schedule (e.g., 3-5 times per week, or another schedule), tracking the inventory of the consumables becomes desirable in order to allow adherence to the prescribed schedule. This may be particularly useful when the dialysis system is located at the home of the patient or some other location removed from a supply center. The dialysis system disclosed tracks the inventory of consumables for the patient, and may communicate with a remote device, also referred to herein as a supply server, to assist the patient in maintaining a sufficient consumables inventory for a prescribed treatment regimen. This may alleviate a burden on the patient to ensure that the necessary materials are present when a prescribed dialysis treatment is scheduled and/or desired.

The dialysis system is configured to track the consumables inventory based on use of the consumables by an individual patient during one or more dialysis treatments, such that the consumables inventory is responsive to the dialysate prescription, the prescribed dialysis treatment schedule, and the duration of dialysis treatments received by the individual patient. Due to differences in the dialysate and schedule prescribed among individual patients, as well as physiological differences among the individual patients themselves, the consumables inventory may decrease at differing rates between individual patients. Further, the dialysis treatment received by an individual patient may vary from one dialysis session to the next based patient response during the treatment or for other reasons, such that the rate of consumable consumption may likewise vary from dialysis treatment session to session. Hence, tracking inventory using a determined expenditure of consumables based on the individual dialysis treatments delivered to the individual patient by the dialysis system may provide more accurate consumables inventory management. The dialysis systems described herein are configured to determine an expenditure of consumables based on the individual dialysis treatments received, such that the dialysis systems are configured to track the consumables inventory in a manner specific to the individual patient. This may provide advantage compared to systems which might track inventory based solely on a schedule of dialysis treatments, without consideration of the actual dialysis delivered during the scheduled treatment.

The dialysis system disclosed is configured to determine a quantity of materials consumed during a dialysis treatment based on an amount of dialysate (“dialysate amount”) provided to the dialyzer over the course of the dialysis treatment. For example, the dialysis system can be configured to determine a dialysate amount indicative of a quantity of dialysate which passes from a dialyzer inlet to a dialyzer outlet over the course of the dialysis treatment. (e.g., as the dialysate circuit replenishes and recycles the dialysate). In examples, the dialysis system (e.g., processing circuitry) determines the dialysate amount by at least determining a flow rate provided by a dialysate pump and substantially integrating the flow rate over the time period of the dialysis treatment. The dialysis system is configured to determine a quantity of one or more of the consumables utilized during the dialysis session based on the dialysate amount determined and the dialysate prescription of the patient. A dialysate prescription can, for example, specify information on the concentration of electrolytes and glucose to be used in a dialysate.

For example, the dialysis system may be configured to determine a quantity of the dialysate chemicals utilized to replenish the dialysate during a dialysis treatment based on the dialysate amount and the prescription. The dialysis system may be configured to determine a quantity of the recharge chemicals necessary to recharge the cartridge following the dialysis treatment based on the dialysate amount and the prescription, e.g., based on predetermined associations between actual dialysate amounts consumed, expected usage of dialysate (as indicated by the prescription), and quantity of recharge chemicals. The dialysate system may be configured to determine a remaining capacity (e.g., ion exchange capacity) of the cartridge based on the dialysate amount and the prescription and, in some examples, also determine the remaining lifetime (e.g., the remaining number of times the cartridge can be recharged and reused for treating the patient). The dialysis system may be configured to track an inventory of consumables remaining and available to the patient based on the dialysate amount and prescription utilized over one or more dialysis treatments, such that the inventory of consumables available to the patient is responsive to potential variations in consumable consumption that may occur from one dialysis treatment session to the next. Again, the variations may be attributable to one or more of patient physiology, which may impact the ultrafiltration volume for a dialysis treatment and may day-to-day consumption depending on how much extra fluid needs to be removed from the patient.

The dialysis system includes processing circuitry configured to determine the dialysate amount. In examples, the circuitry is configured to communicate with a dialysate pump within the dialysate circuit to determine the dialysate amount actually used during a dialysis treatment. The circuitry may be configured to track the consumption of consumables using the dialysate amount and a dialysate prescription for the patient. In examples, the circuitry is configured to receive the dialysate prescription via an input device, such as a card reader, user interface (e.g., a touchscreen display, keypad, and the like), from another device via wired or wireless communication, or other input device. In examples, the circuitry is configured to receive the dialysate prescription via a server or other device (e.g., a tablet). The processing circuitry may be configured to track an inventory of consumables specific to a patient and issue a communication (e.g., to a supply server) when the inventory of consumables falls to be at or below or is anticipated to fall below a threshold consumables inventory for the patient. The supply server may schedule a delivery of the needed consumables to the location of the patient based on the communication from the circuitry of the dialysis system, such that the patient maintains a sufficient inventory of consumables necessary to conduct a prescribed treatment regimen. In examples, when the patient receives the dialysis treatment at a first location and the consumables are delivered from a supply center at a second location geographically displaced from the first location, the supply server schedules a delivery of consumables based on the communication from the dialysis system, the first location of the patient, and the second location of the supply center. The first and second locations may, for example, impact an amount of time required to ship the consumables from the second location to the first location and the supply server and/or the processing circuitry may take anticipated ship times into consideration when scheduling the delivery of consumables.

The dialysis system (e.g., processing circuitry) may be configured to determine a quantity of dialysate chemicals consumed during a dialysis treatment. In examples, the dialysis system is configured to add a solution of one or more dialysate chemicals (also referred to herein as constituent components) to the dialysate (e.g., subsequent to the spent dialysate passing through the cartridge) prior to returning the replenished dialysate to the dialyzer. For example, the dialysis system may be configured to add an infusate solution comprising one or more ions (e.g., calcium, magnesium, and/or potassium ions) subsequent to removal of the ions by the cartridge. The dialysis system may be configured to add a pH control solution (e.g., sodium bicarbonate) subsequent to removal of the ions by the cartridge. The dialysis system may be configured to create different solutions using these dialysate chemicals to accommodate different patient prescriptions. Thus, rather than relying on premixed dialysate solutions (e.g., provided by a manufacturer in dialysate bags), the dialysis system can be configured to create different dialysate solutions using constituent components (which can be part of the consumables described herein in some examples). The dialysis system may be configured to determine a quantity of dialysate chemicals consumed during a dialysis treatment using the dialysate amount which passed through the dialyzer and the prescription of the patient. The dialysis system may track the inventory of dialysate chemicals available at the patient's location based on the quantity of dialysate chemicals consumed. In examples, the dialysis system is configured to generate a solution of a dialysate chemical from a from a solid or powder comprising the dialysate chemical, and the dialysis system is configured to track an inventory of the solid or powder.

The dialysis system (e.g., processing circuitry) may be configured to determine a quantity of recharge chemicals consumed during the recharging of one or more cartridges. While sorbent cartridges are primarily referred to herein, in other examples, the cartridge can be another type of cartridge. Example recharge chemicals include, for example, brine and sodium hydroxide (NaOH). The type of recharge chemicals may depend on the type of cartridge, for example, on whether the cartridge is a cation exchanger or an anion exchanger. In some examples, the sorbent cartridge includes one or more sorbents configured to remove the one or more ions from the spent dialysate passing through the sorbent cartridge during the dialysis treatment. The sorbents may be configured to exchange ions with the spent dialysate, such that the sorbent cartridge generates one or more spent sorbents during the dialysis treatment. The dialysis system may be configured to recharge the cartridge following the dialysis treatment using recharge chemicals (e.g., in a wash fluid) to, for example, substantially restore the ion exchange capacity of the sorbent. This may be referred to as a recharge session. In examples, the dialysis system is configured such that the cartridge can be detached from a dialysate circuit of a dialysis machine and placed in a recharger configured to recharge the sorbent cartridge.

The dialysis system (e.g., processing circuitry) may be configured to determine a quantity of recharge chemicals consumed during the recharging of the sorbent cartridge based on the dialysate amount which passed through the dialyzer and the prescription of the patient (e.g., based on the quantity and composition of spent dialysate to which the sorbent was exposed). The dialysis system may track the inventory of recharge chemicals available at the patient's location based on the quantity of recharge chemicals consumed. In some examples, the volume of recharge chemicals used during a recharge session is constant for all recharge sessions for the particular type of cartridge, such that the quantity of recharge chemicals consumed may be a function of the number of recharge sessions. In examples, the dialysis system is configured to generate a solution of a recharge chemical from a solid or powder comprising the recharge chemical, and the dialysis system is configured to track an inventory of the recharge chemical solid or powder.

The dialysis system (e.g., the processing circuitry of the dialysis system) may be configured to monitor the status of one or more sorbent cartridges utilized during a dialysis treatment. For example, the dialysis system may be configured to utilize one or more cartridges in the dialysate circuit to replenish the dialysate during the treatment. A cartridge may have a defined usable capacity. For example, the cartridge may be a single use cartridge, or may be a multi-use cartridge in which case the cartridge may be recharged using recharge chemicals. The dialysis system may be configured to track the remaining lifetime of a cartridge (e.g., the remaining number of times the cartridge can be recharged and reused) and, in some cases, determine the remaining capacity e.g., the remaining defined usable capacity of the sorbent cartridge) of the cartridge based on the dialysate amount which passed through the dialyzer, a prescription of the patient, and/or the defined usable capacity.

For example, the one or more cartridges may include single use cartridges configured to be substantially discarded after a single dialysis treatment without recharging. The dialysis system may be configured to detect a use of the single use cartridge and track the inventory of single use cartridges in the inventory available to the patient (e.g., at the home of the patient). The one or more sorbent cartridges may include a reusable cartridge configured to be recharged following a dialysis treatment, as discussed above. In examples, a reusable cartridge is configured to be substantially discarded following a limited number of recharges, a set period of time, after an expiration date, or based on some other use limitation, and the dialysis system is configured to track the status of the reusable cartridge. The dialysis system (e.g., the circuitry of the dialysis system) may be configured to substantially monitor the status of each cartridge in a consumables inventory and remove the cartridge from the consumables inventory when the cartridge exceeds the use limitation (e.g., a single use, a number of recharges, a time limit, an expiration date, and/or another use limitation).

The dialysis system may be configured to identify a specific cartridge among the one of more cartridges using a unique cartridge identifier, such as a radio frequency identification (RFID) tag or other machine-readable identifier attached to the specific cartridge. The dialysis system may include one or more readers (e.g., an RFID reader) configured to identify the cartridge identifier. In examples, the processing circuitry of the dialysis system is configured to communicate with a reader to identify a specific cartridge. The circuitry may be configured to substantially monitor and/or update a status of the specific cartridge using the cartridge identifier. For example, the circuitry may be configured to identify a single use cartridge (e.g., using the reader) during a dialysis treatment and update the inventory of consumables based on the single use. The circuitry may be configured to track a number of times a reusable sorbent cartridge has been recharged, a time period over which the reusable sorbent cartridge has been utilized, an associated expiration date of the sorbent cartridge, or proximity to another use limitation, and update the inventory of consumables when the use limitation indicates the sorbent cartridge should be removed from the inventory.

Thus, the dialysis system is configured to determine a quantity of materials consumed during a dialysis treatment based on an amount of dialysate provided to the dialyzer over the course of a dialysis treatment and a dialysate prescription of the patient. The dialysis system may be configured to determine a quantity of dialysate chemicals, recharge chemicals, and/or remaining lifetime or capacity of a sorbent cartridge based on the dialysate amount and the dialysate prescription. The dialysis system may track an inventory of consumables using the quantity of materials consumed, such that the inventory of consumables tracked is specific to an individual patient. The dialysis system may issue a communication to a supply server, such that the supply server may schedule a delivery to the location of the patient.

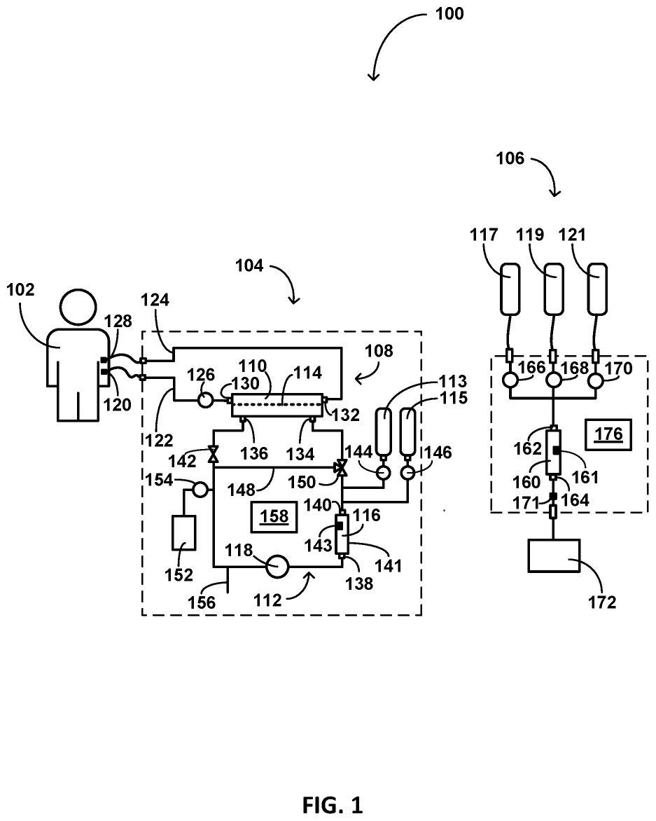

FIG. 1 schematically illustrates an example dialysis system 100 configured to deliver a dialysis treatment to a patient 102. Dialysis system 100 includes a dialysis machine 104 and a recharger 106. Dialysis system 100 includes an extracorporeal circuit 108 configured to transfer some portion of the blood of patient 102 through a dialyzer 110. A dialysate circuit 112 of dialysis system 100 is configured to circulate a dialysate through dialyzer 110. Dialyzer 110 includes a membrane 114 substantially separating extracorporeal circuit 108 and dialysate circuit 112, such that waste products are transferred from the blood of the patient to the dialysate as dialysis system 100 transfers the blood of the patent and the dialysate through dialyzer 110.

Dialysate circuit 112 is configured to replenish and recirculate the dialysate through dialyzer 110 over the course of a dialysis treatment (e.g., as blood of patient transfers through extracorporeal circuit 108). In examples, dialysate circuit 112 is configured to receive a spent dialysis from dialyzer 110 and transfer the spent dialysate to a sorbent cartridge 116 using a dialysate pump 118. Sorbent cartridge 116 is configured to remove at least some portions of the waste products in the spent dialysate (e.g., using one or more sorbents). In examples, dialysate circuit 112 is configured to add one or more dialysate chemicals (e.g., in the form of an infusate solution and/or pH control solution) to the dialysate via container 113 and/or container 115. Dialysate circuit 112 may be configured to transfer the thus replenished dialysate (e.g., using dialysate pump 118) from sorbent cartridge 116 back to dialyzer 110, such that dialysate circuit 112 replenishes and recirculates the dialysate through dialyzer 110 as extracorporeal circuit 108 transfers the blood of the patient through dialyzer 110.

Sorbent cartridge 116 may include one or more sorbents configured to sorb (e.g., absorb or adsorb) waste products from the spent dialysate discharging from dialyzer 110. For example, the one or more sorbents may be configured to exchange ions with the spent dialysate to remove the waste products. Sorbent cartridge 116 may thus have a usable capacity (e.g., a functional capacity) for removal of waste products based on the sorbing capacity of the one or more sorbents. In the example shown in FIG. 1, dialysis system 100 is configured to recharge sorbent cartridge 116 to substantially restore the usable capacity of sorbent cartridge 116, such that sorbent cartridge 116 may be utilized in a subsequent dialysis treatment. In other examples, cartridge 116 is a single-use cartridge that is not rechargeable and system 110 may not include recharger 106.

Recharger 106 is configured to recharge sorbent cartridge 116 to substantially restore its usable capacity using one or more recharge chemicals. Recharger 106 may be configured as a separate component having a physically separate housing from dialysis machine 104 (as illustrated in FIG. 1), or may be configured such that one or more components of recharger 106 are mechanically supported within a housing common to one or more components of dialysis machine 104. Sorbent cartridge 116 may be configured such that a clinician and/or patient or patient caretaker may disconnect sorbent cartridge 116 from dialysis machine 104 following a dialysis treatment and connect sorbent cartridge 116 to recharger 106 for recharging in preparation for a subsequent dialysis treatment. Recharger 106 is configured to expose the one or more sorbents to the recharge chemicals (e.g., in the form of a basic solution, an acidic solution, and/or heated water) to restore some or all of the usable capacity of sorbent cartridge 116. For example, recharger 106 may include one or more containers such as container 117, container 119, and/or container 121 configured to hold one or more solutions of the recharge chemicals. Recharger 106 may be configured to flow the one or more solutions through sorbent cartridge 116 to restore some or all of its usable capacity. Following the recharging, sorbent cartridge 116 may be removed from recharger 106 and connected to dialysis machine 104 for subsequent dialysis treatments.

Dialysis system 100 is thus configured to utilize consumable items such as the dialysate chemicals and the recharge chemicals. Further, sorbent cartridge 116 may have a particular capacity (e.g., ion exchange capacity) and/or a recommended lifetime (e.g., a limited number of recharging cycles, an expiration date, and/or some other usage limitation). The amount of dialysate chemicals, recharge chemicals, and capacity of sorbent cartridge 116 consumed over the course of a dialysis treatment and recharge may be dependent on a dialysate prescription and a duration of the dialysis treatment. The duration may be dependent on one or more physiological parameters of the patient (e.g., a patient weight) which may vary between dialysis treatments, such that the amount of dialysate chemicals, recharge chemicals, and capacity of sorbent cartridge 116 consumed may likewise vary between dialysis treatments. Hence, the dialysis consumable inventory available to the patient may decrease at varying rates, depending on the amount of dialysate chemicals, recharge chemicals, and capacity of sorbent cartridge 116 consumed for each dialysis treatment administered using dialysis system 100. Tracking the consumables inventory on a per-treatment basis to ensure a satisfactory quantity of consumables are on hand to conduct prescribed dialysis treatments may place a burden on a clinician and/or patient.

Extracorporeal circuit 108 of dialysis machine 104 may be configured to deliver blood of patient 102 to dialyzer 110 to enable a transfer of waste products from the blood to a dialysate. A shunt 120 such as a needle, cannula, or catheter may be fluidically connected to vasculature of patient 102 to withdraw and transfer the blood of the patient to extracorporeal circuit 108. In examples, extracorporeal circuit 108 includes an arterial line 122 configured to transport blood from patient 102 and a venous line 124 configured to return blood to the patient via, for example, shunt 128. Extracorporeal circuit 108 may include a blood pump 126 configured to transfer the blood of the patient through dialyzer 110 and extracorporeal circuit 108. Blood pump 126, under the control of control circuitry of dialysis system 100, may be configured to adjust to provide a required flow rate suitable for the dialysis treatment. In examples, blood pump 126 conveys blood through dialyzer 110 to enable a transfer of waste products from the blood of patient 102 to the dialysate provided by dialysate circuit 112. In examples, the blood of patient 102 enters the dialyzer 110 through an inlet 130 of dialyzer 110 (“blood inlet 130”) and exits through an outlet 132 of dialyzer 110 (“blood outlet 132”).

In some examples, dialysis machine 104 includes one or more sensors configured to indicate and/or monitor an adequacy of blood flow within extracorporeal circuit 108 and/or dialyzer 110. Dialysis machine 104 may be configured (e.g., using one or more pressure meters (not shown)) to determine a pressure of blood entering blood inlet 130, a pressure at blood exiting blood outlet 132, a differential pressure from blood inlet 130 to blood outlet 132, and/or other parameters to indicate and/or monitor the adequacy of the blood flow. In examples, extracorporeal circuit 108 is configured to help prevent the introduction of air into the circulatory system of patient 102 (e.g., using an air trap (not shown)). Dialysis machine 104 may be configured to monitor extracorporeal circuit 108 (e.g., using one or more air-fluid detectors (not shown)) to confirm that air is not appreciably present in extracorporeal circuit 108. In examples, extracorporeal circuit 108 is configured to add one or more additives (e.g., an anticoagulant) to the blood within extracorporeal circuit 108 to prevent clotting within the dialyzer 110 and/or extracorporeal circuit 108. In examples, dialysis machine 104 is configured to determine a hematocrit level of the blood within extracorporeal circuit 108 using, for example a hematocrit sensor (not shown).

Dialysate circuit 112 is configured to convey dialysate to a dialysate side of dialysis membrane 114 to remove waste products from the blood transferred through extracorporeal circuit 108. In the example shown in FIG. 1, dialyzer 110 defines an inlet 134 (“dialyzer inlet 134”) and an outlet 136 (“dialyzer outlet 136”). Dialysate pump 118 may be configured to provide a motive force to cause the dialysate to enter dialyzer 110 at dialyzer inlet 134 and exit dialyzer 110 at dialyzer outlet 136. Dialysate conveyed to dialyzer 110 may remove the waste products by diffusion, hemofiltration, hemodiafiltration, and/or some other method using membrane 114. In examples, dialysate circuit 112 is configured to receive the spent dialysate from dialyzer outlet 136 and transfer the spent dialysate to an inlet 138 of sorbent cartridge 116 (“cartridge inlet 138”). Sorbent cartridge 116 may be configured to discharge the dialysate via an outlet 140 of sorbent cartridge 116 (“cartridge outlet 140”). In examples, dialysate pump 118 is configured to cause the dialysate to enter sorbent cartridge 116 at cartridge inlet 138 and exit sorbent cartridge 116 at cartridge outlet 140.

Sorbent cartridge 116 is configured to remove one or more waste products from the dialysate as the dialysate flows through sorbent cartridge 116. In examples, sorbent cartridge 116 is configured to exchange ions with the dialysate flowing through sorbent cartridge 116. For example, sorbent cartridge 116 may be configured to act as cation exchanger to absorb ammonia ions and other cations (e.g., potassium (K+), calcium (Ca2+), magnesium (Mg2+), and/or others). Sorbent cartridge 116 may include one or more sorbents including one or more sorbing and/or catalyzing materials, such as urease, zirconium phosphate, magnesium phosphate, zirconium oxide material, activated carbon, and/or other materials. In some examples, one or more of the sorbents are arranged into discrete layers within sorbent cartridge 116. In some examples, one or more of the sorbents are substantially intermixed within sorbent cartridge 116. Sorbent cartridge 116 may be configured to define one or more flow paths between cartridge inlet 138 and cartridge outlet 140, such that a dialysate flowing from cartridge inlet 138 to cartridge outlet 140 may fluidly encounter substantially all of the one or more sorbents in some examples, and only a portion of the one or more sorbents in other examples.

Sorbent cartridge 116 may include a cartridge housing 141 defining one or more volumes configured to contain the one or more sorbents. In examples, cartridge housing 141 is configured such that the one or more sorbents are substantially contained within a single volume defined by cartridge housing 141. For example, the one or more sorbents may be arranged substantially in one or more layers or substantially intermixed within the single volume. In some examples, sorbent cartridge 116 defines a plurality of volumes with each volume configured to contain a portion of the one or more sorbents. For example, sorbent cartridge 116 may define a first volume configured to contain a first sorbent. Sorbent cartridge 116 may define a second volume configured to contain a second sorbent. Sorbent cartridge 116 may be configured such that when a dialysate enters cartridge inlet 138 and discharges from cartridge outlet 140, at least some portion of the dialysate flows through the first volume and/or the second volume.

In examples, sorbent cartridge 116 includes a plurality of physically separate modules configured to mechanically connect together to define cartridge housing 141. Each module may define a volume configured to contain a portion of the one or more sorbents. A first module may define the first volume and a second module may define the second volume. In examples, the first module and the second module may be configured to mechanically disconnect such that, for example, the first module and the second module may be individually connected to recharger 106 as separated modules.

In some examples, sorbent cartridge 116 includes one or more cartridge identifiers such as identifier 143 that is unique to cartridge 116. Identifier 143 may be, for example, a radio frequency identification (RFID) tag (storing a unique identification number or the like) or other identifier attached to sorbent cartridge 116. Dialysis system 100 may include one or more readers (e.g., an RFID reader) configured to identify identifier 143 such that, for example, dialysis system 100 may associate sorbent cartridge 116 with identifier 143. In some examples, when sorbent cartridge 116 includes a plurality of modules configured to mechanically connect and/or mechanically disconnect from each other, a particular module may include a unique identifier such as identifier 143. Dialysis system 100 may be configured to associate the particular module with the unique identifier.

Dialysate circuit 112 is configured to add one or more dialysate chemicals to the dialysate prior to returning the dialysate to dialyzer 110. Example dialysate chemicals include glucose and electrolytes. In some examples, the dialysate chemicals are added to increase a concentration of ions in the dialysate. The dialysate chemicals may be added to increase a concentration of ions removed by sorbent cartridge 116. For example, sorbent cartridge 116 may be configured to remove potassium, calcium, magnesium, and/or other cations as the dialysate flows from cartridge inlet 138 to cartridge outlet 140. Dialysate circuit 112 may be configured to add potassium, calcium, magnesium, and/or the other cations (e.g., in an infusate solution) to compensate for the removal prior to returning the dialysate to dialyzer 110. In some examples, dialysate circuit 112 is configured to add dialysate chemicals to control a pH of the dialysate. For example, dialysate circuit 112 may be configured to add a bicarbonate or other salt for control of pH. Dialysate circuit 112 may be configured to add the dialysate chemicals at a substantially continuous rate, a varying rate, and/or substantially as a batch addition. Dialysate circuit 112 may be configured to add the dialysate chemicals in any manner and in any location of dialysate circuit 112. In examples, dialysate circuit 112 includes one or more containers such as container 113 and container 115 configured to hold one or more dialysate chemicals. Dialysate circuit 112 may include one or more pumps such as pump 144 and pump 146 configured to transfer the one or more dialysate chemicals to the dialysate.

In examples, dialysate circuit 112 includes a bypass line 148 configured to define a flow path for the dialysate that bypasses dialyzer 110. Dialysate circuit 112 may be configured to cause dialysate to flow through bypass line 148 based on a sensed condition of the dialysate (e.g., a conductivity or other sensed condition), or for other reasons. In examples, dialysate circuit 112 may include a valve 150 (e.g., a three-way valve) configured to cause the dialysate to flow through bypass line 148. Dialysate circuit 112 may include a valve 142 configured to control (e.g., commence and/or cease) a flow of dialysate through dialyzer 110. In some examples, dialysis system 100 includes a control reservoir 152 configured to withdraw and/or inject dialysate (e.g., using pump 154) into the dialysate circulating among dialyzer 110, pump 118, cartridge 116, and/or bypass line 148. Control reservoir 152 may be configured to withdraw and/or inject dialysate to, for example, control a pressure of the dialysate circulating among dialyzer 110, pump 118, cartridge 116, and/or bypass line 148.

In some examples, dialysis machine 104 includes one or more sensors configured to indicate and/or monitor an adequacy of dialysate flow within dialysate circuit 112 and/or dialyzer 110. For example, dialysis machine 104 can be configured to monitor a conductivity of the dialysate within dialysate circuit 112 using, for example, one or more conductivity sensors (not shown). As another example, dialysis machine 104 may be configured to monitor and/or determine a pressure within dialysate circuit 112 using, for example, one or more pressure sensors (not shown). Dialysis machine 104 may be configured to substantially determine a presence of blood in the dialysate indicating, for example, a breach in membrane 114 using, for example, one or more blood leak detectors (not shown). Dialysis machine 104 may be configured to substantially confirm that air is not appreciably present in dialysate circuit 112 using, for example, one or more air-fluid detectors (not shown)). In some examples, dialysis machine 104 is configured to add a fluid such as water to dialysate circuit 112 using, for example, a fluid connection 156 configured to fluidly connect dialysate circuit 112 to a fluid source, such as a fluid reservoir and/or a fluid pump.

Dialysis system 100 includes processing circuitry 158 configured to control one or more components of dialysis machine 104 and/or monitor the blood of the patient within extracorporeal circuit 108 and/or the dialysate within dialysate circuit 112. In examples, processing circuitry 158 is configured to control one or more pumps such as dialysate pump 118, blood pump 126, pump 144, 146, and/or other pumps within dialysis system 100. In some examples, processing circuitry 158 is configured to determine a volume of fluid being removed from the circulation of patient 102 and/or being infused into the circulation of patient 102 using, for examples, the hematocrit sensor. In addition, in some examples, processing circuitry 158 may be configured to determine an amount of sodium substantially removed from patient 102 during a treatment based on, for example, conductivity of the blood within extracorporeal circuit 108 and/or the volume of fluid removed or infused. Processing circuitry 158 may be configured to monitor a pH of the dialysate within dialysate circuit 112 and control the addition of dialysate chemicals based on the pH. Processing circuitry 158 may be configured enable the flow of dialysate through bypass line 148 (e.g., based on a sensed conductivity of the dialysate) using, for example, valve 150 and/or valve 142. Processing circuitry 158 may be configured to withdraw and/or inject dialysate (e.g., using pump 154) into the dialysate circulating among dialyzer 110, pump 118, cartridge 116, and/or bypass line 148 based on, for example, a pressure of the circulating dialysate. Processing circuitry 158 may be configured to control one or more components of dialysis system 100 in any manner necessary to perform the functions of dialysis system 100 described herein. Any of the aforementioned functions of processing circuitry 158 can be used alone or in combination with each other.

As discussed, the amount of dialysate chemicals (e.g., from containers 113, 115) and/or the capacity of sorbent cartridge 116 consumed over the course of a dialysis treatment may be dependent on a dialysate prescription and a duration of the dialysis treatment. Dialysis system 100 (e.g., processing circuitry 158 and/or other processing circuitry) may be configured to determine the amount of dialysate chemicals and/or the capacity of sorbent cartridge 116 consumed using a dialysate amount indicative of an actual amount of dialysate which passed from dialyzer inlet 134 to dialyzer outlet 136 over the course of the dialysis treatment (e.g., as dialysate circuit 112 replenishes and recycles the dialysate). In examples, dialysis system 100 determines the dialysate amount by at least measuring a flow rate provided by dialysate pump 118 and substantially integrating the flow rate over a time period of the dialysis treatment. Dialysis system 100 may treat the dialysate amount as indicative of the amount of dialysate chemicals and the capacity of sorbent cartridge 116 consumed during the dialysis treatment.

In some examples, dialysis system 100 is configured to determine a bypass quantity of dialysate which flowed through bypass line 148 during the treatment and account for (e.g., substantially subtract) the bypass quantity when determining the integrated flow rate of dialysate pump 118. In some examples, dialysis system 100 determines the dialysis amount using other parameters monitored during a dialysis treatment, such as flow meter configured to monitor a flow through dialyzer 110, one or more pressure sensors configured to monitor a differential pressure across dialyzer 110, and/or other parameters indicative of a dialysate flow through dialyzer 110.

Dialysis system 100 (e.g., processing circuitry 158 and/or other processing circuitry) may be configured to associate the determined dialysate amount with sorbent cartridge 116 and/or one or more modules comprising sorbent cartridge 116. In examples, dialysis system 100 is configured to associate the dialysate amount and a dialysate prescription with sorbent cartridge 116 and/or one or more modules comprising sorbent cartridge 116. Dialysis system 100 may be configured to associate the dialysate amount and a dialysate prescription with sorbent cartridge 116 and/or one or more modules comprising sorbent cartridge 116 using, for example, identifier 143.

Recharger 106 is configured to recharge a sorbent cartridge 160 to restore some or all of a usable capacity (e.g., following a dialysis treatment provided by dialysis machine 104) using one or more recharge chemicals. Sorbent cartridge 160 may be the same or a different sorbent cartridge from sorbent cartridge 116, or may be a first module, a second module, or another portion of sorbent cartridge 116. Sorbent cartridge 160 includes a one or more cartridge identifiers such as identifier 161. Identifier 161 may be, for example, a RFID tag or other identifier attached to sorbent cartridge 160 that uniquely identifies a particular cartridge 160. Recharger 106 may include one or more readers (e.g., an RFID reader) configured to identify identifier 161 such that, for example, dialysis system 100 may associate sorbent cartridge 160 with identifier 161. In examples, sorbent cartridge 160 is an individual module configured to mechanically connect and/or mechanically disconnect with other modules to define a sorbent cartridge, and dialysis system 100 is configured to associate the individual module with identifier 161.

Recharger 106 is configured to expose the one or more sorbents to one or more recharge chemicals (e.g., in the form of a basic solution, an acidic solution, heated water, and/or other fluids) to restore some or all of the usable capacity of sorbent cartridge 160. In some cases, recharging may refer to the process of treating the one or more sorbents within sorbent cartridge 160 to restore the usable capacity (e.g., functional capacity) of the one or more sorbent materials, such that the sorbent materials are restored to a condition for use or reuse in a subsequent dialysis treatment. Recharging may include treating the one or more sorbents to substantially clean the one or more sorbents. The total mass, weight and/or amount of the one or more sorbents may alter or remain substantially the same as a result of the recharging. In examples, recharging may involve exchanging ions bound to the one or more sorbents with different ions.

Recharger 106 may include one or more containers such as container 117, container 119, and/or container 121 configured to hold a solution of the one or more recharge chemicals. Recharger 106 may be configured to recharge the one or more sorbents within a sorbent cartridge 160 using the recharge chemicals. In examples, recharger 106 is configured to fluidly connect the one or more sorbents within sorbent cartridge 160 and the recharge chemicals held by container 117, 119, 121 when sorbent cartridge 160 is connected to recharger 106. In examples, recharger 106 is configured such that the solution of the one or more recharge chemicals flows from container 117, 119, 121 into an inlet 162 of sorbent cartridge 160 (“cartridge inlet 162”) and discharges from an outlet 164 of sorbent cartridge 160 (“cartridge outlet 164”). Recharger 106 may include one or more recharging pumps such as recharging pump 166, recharging pump 168, and/or recharging pump 170 configured to provide a motive force to a fluid flowing from one of container 117, 119, 121, respectively, to sorbent cartridge 160. Recharger 106 may include a flow sensor 171 configured to detect and/or measure a flow through sorbent cartridge 160. In examples, recharger 106 includes a waste reservoir 172 configured to receive and/or collect fluid discharged from sorbent cartridge 160 (e.g., discharged from cartridge outlet 164).

In some examples, recharger 106 is configured to recharge a first sorbent using a first recharging chemical and recharge a second sorbent using a second recharging chemical. For example, recharger 106 may be configured to fluidly connect a first module containing a first sorbent with container 117 such that the first sorbent may be recharged using the first recharging chemical. Recharger 106 may be configured to fluidly connect a second module containing a second sorbent with container 119, such that the second sorbent may be recharged using the second recharging chemical. Recharger 106 may be configured to fluidly connect any number of sorbent cartridges and/or any number of modules with one or more of container 117, container 119, and/or container 121, or other containers holding one or more recharge chemicals. In some examples, recharger 106 may be configured to recharge the one or more sorbents using the first recharging chemical and the second recharging chemical sequentially. For example, recharger 106 may be configured to initially fluidly connect sorbent cartridge 160 with container 117 to at least partially recharge the one or more sorbents using the first recharging chemical, then subsequently fluidly connect sorbent cartridge 160 with container 119 to at least partially recharge the one or more sorbents using the second recharging chemical.

In some examples, recharger 106 is configured to deliver one or more of the recharge chemicals to sorbent cartridge 160 as a recharging solution and/or deliver one or more recharge chemicals as an acidic solution to sorbent cartridge 160. The one or more sorbents of sorbent cartridge 160 may include zirconium phosphate or another sorbent capable of recharging when exposed to the acidic solution. Recharger 106 may be configured to deliver one or more recharge chemicals as a basic solution to sorbent cartridge 160. The one or more sorbents of sorbent cartridge 160 may include zirconium oxide or another sorbent capable of recharging when exposed to the basic solution. In examples, the recharge chemicals include a catalyst such as urease, and recharger 106 is configured to deliver the catalyst as a catalyst-containing solution to sorbent cartridge 160. The one or more sorbents of sorbent cartridge 160 may include alumina or another sorbent capable of recharging when exposed to the catalyst-containing solution.

Dialysis system 100 includes processing circuitry 176 configured to communicate with and/or control one or more components of recharger 106, such as recharging pump 166, recharging pump 168, recharging pump 170, flow sensor 171, and/or other components within dialysis system 100. Processing circuitry 176 may be configured to control the components of recharger 106 to substantially control an amount of a recharging solution delivered to sorbent cartridge 160. Processing circuitry 176 may be configured to control one or more components of dialysis system 100 in any manner necessary to perform the functions of dialysis system 100 described herein.

In some examples, dialysis system 100 (e.g., processing circuitry 176 or other processing circuitry) is configured to determine an amount of recharge chemicals necessary to recharge sorbent cartridge 160 based on a preceding dialysis treatment conducted using sorbent cartridge 160. Dialysis system 100 may be configured to determine the amount of recharge chemicals using the dialysate amount determined for one or more most recent preceding dialysis treatments. For example, dialysis system 100 may be configured to identify sorbent cartridge 160 using identifier 161 and determine that sorbent cartridge 160 was utilized in a preceding dialysis treatment administered by dialysis machine 104. Dialysis system 100 may be configured to determine the preceding dialysis treatment resulted in a particular dialysate amount (e.g., a dialysate amount indicative of an amount of dialysate transferred through dialyzer 110 during the preceding dialysis treatment). Dialysis system 100 may be configured to determine an amount of recharge chemicals consumed and/or anticipated to be consumed to recharge sorbent cartridge 160 using the dialysate amount and the dialysate prescription. In some examples, dialysis system 100 is configured to recharge sorbent cartridge 160 using a specified amount of recharge chemicals after each dialysis treatment where sorbent cartridge 160 is used. Dialysis system 100 may be configured to determine an amount of recharge chemicals consumed and/or anticipated to be consumed using the specified amount.

Thus, dialysis system 100 may be configured to determine a quantity of dialysate chemicals consumed during a dialysis treatment and/or a quantity of recharge chemicals consumed during the recharging of one or more sorbent cartridges. Dialysis system 100 may be configured to determine a quantity of dialysate chemicals consumed and/or a quantity of recharge chemicals consumed based on a dialysate prescription and a dialysate amount, where the dialysate amount is indicative of an amount of dialysate which passed from dialyzer inlet 134 to dialyzer outlet 136 over the course of the dialysis treatment. Dialysis system 100 may receive the dialysate prescription from a user or another device. Dialysis system 100 may determine the dialysate amount based on an integrated flow rate of dialysate pump 118, and/or based on other parameters monitored during the dialysis treatment.

Dialysis system 100 may be configured to determine a quantity of dialysate chemicals consumed by dialysis machine 104 (e.g., added to dialysate circuit 112 via container 113, 115) during delivery of the dialysis treatment using the dialysate prescription and the dialysate amount. Dialysis system 100 may be configured to determine a quantity of recharge chemicals consumed by recharger 106 (e.g., via container 117, 119, 121) during a subsequent recharge after the dialysis treatment using the dialysate prescription and the dialysate amount. Dialysis system 100 may be configured to determine a remaining capacity of a sorbent cartridge (e.g., sorbent cartridge 116 and/or sorbent cartridge 160) using the dialysate prescription and the dialysate amount. In examples, dialysis system 100 is configured to associate the dialysate prescription and/or dialysate amount with a given sorbent cartridge (e.g., sorbent cartridge 116, 160) using a unique identifier (e.g., identifier 143, 161) such that, for example, the remaining capacity of the given sorbent cartridge may be tracked over one or more dialysis treatments, and/or the quantity of recharge chemicals anticipated to be consumed during a recharging may be determined.

In addition to or instead of tracking the capacity of cartridge, in some examples, dialysis system 100 (e.g., processing circuitry) is configured to determine a remaining lifetime of the cartridge by at least tracking the number of times the cartridge has been used for treatment (and subsequently recharged). Some cartridges, for example, may only be used and recharged 10-20 times, such as 16 times. For example, dialysis system 100 can associate the cartridge (e.g., sorbent cartridge 116, 160) using a unique identifier (e.g., identifier 143, 161) and associate the number of uses with the unique identifier, such that the used or remaining lifetime of a given cartridge may be tracked. This may enable system 100 to track the future cartridge needs of the patient to facilitate reordering of cartridges.

Dialysis system 100 may track an inventory of consumables available to a patient based on the dialysate chemicals consumed, the recharge chemicals consumed, and/or the remaining lifetime of one or more cartridges used by the patient, and may communicate with a remote device, such as a supply server, to assist the patient in maintaining a sufficient consumables inventory for a prescribed treatment regimen.

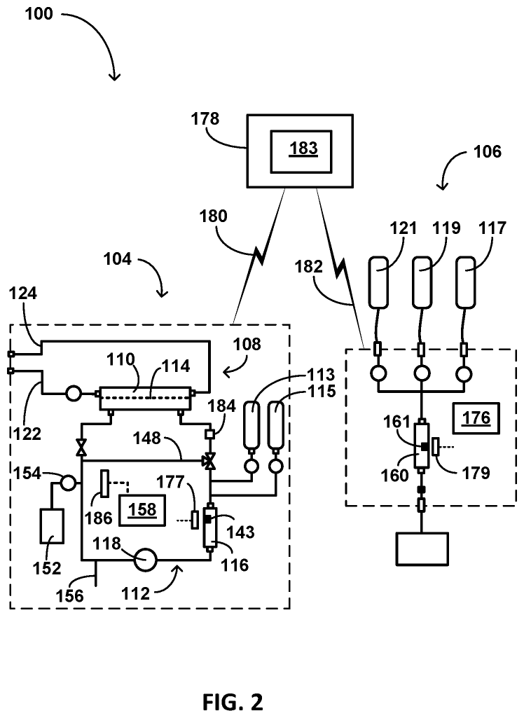

FIG. 2 illustrates further details of dialysis system 100, which includes dialysis machine 104 and recharger 106, and also illustrates an example supply server 178. Dialysis machine 104 is configured to determine a quantity of materials consumed during a dialysis treatment delivered by dialysis machine 104 and/or a recharge conducted by recharger 106, such as a quantity of dialysate chemicals, a quantity of recharge chemicals, a quantity of sorbent cartridges, and/or a portion of a capacity or lifetime of a sorbent cartridge. Dialysis system 100 may be configured to track an inventory of consumables based on the determined quantity of materials consumed. In some examples, dialysis system 100 (e.g., dialysis machine 104 and/or recharger 106) is configured to communicate the quantity of materials consumed to a supply server 178 to, for example, to assist a patient using dialysis system 100 in maintaining a sufficient consumables inventory for a prescribed treatment regimen. Supply server 178 can be, for example, a remote device in a different location than dialysis machine 104. In some examples, dialysis system 100 is configured to track an inventory of consumables based on the quantity of materials consumed and communicate the inventory of consumables to supply server 178.

In the example shown in FIG. 2, processing circuitry of dialysis system 100 is configured to communicate with supply server 178 using communication circuitry. For example, processing circuitry 158 can be configured to communicate with supply server 178 via communication link 180) and/or processing circuitry 176 can be configured to communicate with supply server 178 via communication link 182. Supply server 178 may comprise one or more servers, a cloud, one or more databases, and processing circuitry configured to implement functionality and/or process instructions for execution within supply server 178. For example, as shown in FIG. 2, supply server 178 may include processing circuitry 183 configured to perform functions attributed to supply server 178 herein.

One or more of processing circuitry 158, 176, or 183 may be configured to identify sorbent cartridge 116, 160 using a reader, such as an RFID reader or other reader configured to identify sorbent cartridge 116, 160 using the respective identifier 143, 161. For example, dialysis machine 104 may include reader 177 configured to identify sorbent cartridge 116, 160. In addition or instead, recharger 106 may include reader 179 configured to identify sorbent cartridge 116, 160.

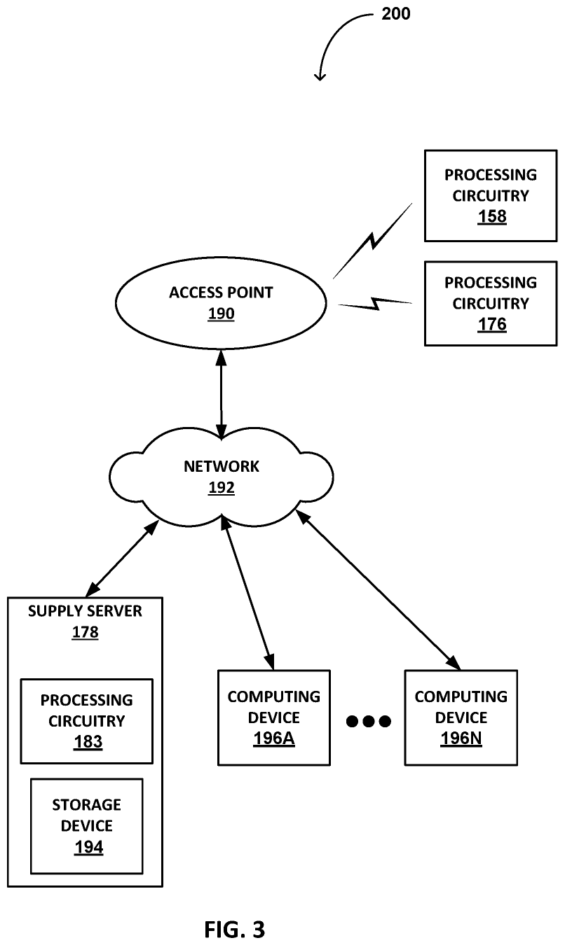

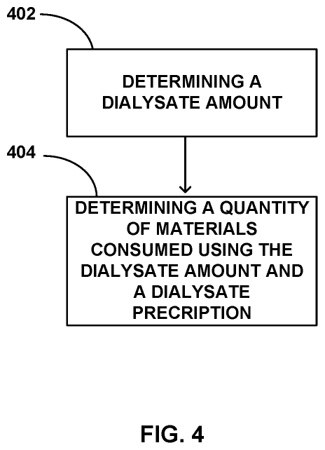

Although processing circuitry 158 is primarily referred to in the description of the remainder of FIG. 2, as well as FIGS. 3 and 4, the description can also apply to processing circuitry 176, 183, and/or other processing circuitry of system 100 in other examples. Thus, the description of processing circuitry 158 may apply to processing circuitry 176, 183, or other processing circuitry of system 100 herein. In other examples, processing circuitry 176, 183, or other processing circuitry, alone or in combination with processing circuitry 158 can perform any of the functions attributed to processing circuitry 158 herein.

Processing circuitry 158 is configured to determine the quantity of materials consumed during one or more dialysis treatments using a dialysate amount. The dialysate amount is indicative of an amount of dialysate (e.g., a mass and/or volume) which passes through dialyzer 110 over the course of a dialysis treatment. The dialysate amount may be indicative of a flow rate of dialysate through dialyzer 110 (e.g., as the dialysate circulates in dialysate circuit 112) substantially integrated over a time period over which the flow rate occurs (e.g., a over a dialysis treatment). In some examples, processing circuitry 158 is configured to determine a dialysate amount based on the operation of dialysate pump 118, as dialysate pump 118 provides motive force to circulate the dialysate through dialysate circuit 112 and dialyzer 110.

In examples, processing circuitry 158 is configured to determine a flow rate of dialysate provided by dialysate pump 118 based on a pump capacity of dialysate pump 118. The pump capacity may be a volume or mass flow rate of dialysate provided by or anticipated to occur from dialysate pump 118 when dialysate pump 118 causes dialysate to flow through dialysate circuit 112. In some examples, dialysate pump 118 is configured to operate over a plurality of pump speeds, and the pump capacity is dependent on the pump speed. In some examples, the pump capacity is dependent on a flow configuration of dialysate circuit 112 (e.g., whether valve 150 is enabling flow through bypass line 148, whether pump 154 is withdrawing or injecting dialysate from or to reservoir 152, a position of valve 142, or another flow configuration of dialysate circuit 112). In some examples, the pump capacity of dialysate pump 118 may be based on a direct measurement of the flow performance of dialysate pump 118, a flow rating of dialysate pump 118 at one or more pump speeds, and/or some other method of determination. In some example, dialysate pump 118 is a positive displacement pump (e.g., a peristaltic pump, diaphragm pump, and others) having a substantially constant pump capacity at a given speed.

Processing circuitry 158 may be configured to determine a flow rate of dialysate based on the pump capacity of dialysate pump 118. In examples, processing circuitry 158 is configured to determine the dialysate amount based on an operating time of dialysate pump 118 and a pump capacity of dialysate pump 118 during the operation. Processing circuitry 158 may be configured to define the pump capacity (e.g., from pump capacity values stored in a memory) based on an operating speed of dialysate pump 118, a flow configuration of dialysate circuit 112, or other factors impacting the pump capacity of dialysate pump 118. In examples, processing circuitry 158 is configured to communicate with and/or receive communications from one or more components of dialysate circuit 112 (e.g., dialysate pump 118, pump 154, valve 150, and/or valve 142) to define the pump capacity.

In some examples, processing circuitry 158 is configured to determine a flow rate of dialysate based on a direct measurement of the flow rate. For example, dialysis system 100 (e.g., dialysis machine 104) may include one or more flow sensors such as flow sensor 184 configured to detect and/or measure a flow of dialysate through dialysate circuit 112. Flow sensor 184 may be configured to measure a flow rate within dialysate circuit 112 indicative of a flow rate through dialyzer 110. In examples, flow sensor 184 is configured to communicate the flow rate to processing circuitry 158, e.g., by transmitting an electrical signal indicative of the flow rate to processing circuitry 158. Flow sensor 184 may include, for example, a flow meter configured to measure the dialysate flow through a portion of dialysate circuit 112, one or more pressure sensors configured to monitor a differential pressure across a portion of dialysate circuit 112 and/or dialyzer 110, and/or another sensor configured to generate an electrical signal indicative of a parameter of a dialysate flow through dialyzer 110.

Processing circuitry 158 may be configured to determine the dialysate amount based on the flow rate. In examples, processing circuitry 158 is configured to determine the dialysate amount based on the determined flow rate and a time over which the flow rate substantially occurred. Processing circuitry 158 may be configured to determine the time over which the flow rate occurred using an operating time of dialysate pump 118, a communication from flow sensor 184, and/or a communication from another component of dialysis system 100. Processing circuitry 158 may be configured to determine the dialysate amount based on the measured flow rate and the time over which the flow rate occurred.

Processing circuitry 158 is further configured to determine the quantity of materials consumed based on a dialysate prescription. The dialysate prescription may be indicative of one or more dialysate chemicals added to the dialysate within dialysate circuit 112 by dialysis system 100 and/or a composition of the dialysate prescribed to the patient. For example, the prescription may specify the composition of the dialysate when the dialysate flows into or through dialyzer 110, or may specify a composition of the dialysate in some other portion of dialysate circuit 112. Dialysis system 100 (e.g., dialysis machine 104) may be configured to substantially establish and/or maintain the composition of the dialysate using the dialysate chemicals. For example, dialysis system 100 may be configured to substantially maintain the composition of the dialysate in a portion of dialysate circuit 112 by adding (e.g., injecting) the dialysate chemicals (e.g., from container 113, 115) into the dialysate circulating within dialysate circuit 112. In examples, the dialysate chemicals include one or more materials which form an ion in solution, such as a material comprising potassium, calcium, magnesium, and/or the ion-forming materials. In examples, the dialysate chemicals include one or more materials in an electrolytic solution. In some examples, dialysate circuit 112 is configured to add dialysate chemicals to control a pH of the dialysate. For example, dialysate circuit 112 may be configured to add a bicarbonate or other salt for control of pH. In some cases, dialysis system 100 is configured to add the dialysate chemicals subsequent to the dialysate passing through sorbent cartridge 116 and prior to returning the thus replenished dialysate to dialyzer 110.

In examples, processing circuitry 158 is configured to receive the dialysate prescription via an input device, such as input device 186. Input device 186 may be, for example, a card reader, a user interface (e.g., a touchscreen display, a tablet, a keypad, and the like), or other input device configured to receive the dialysate prescription. In addition or instead, processing circuitry 158 is configured to receive the dialysate prescription from another device via communication circuitry, e.g., from supply server 178 or a remote clinician computer. For example, supply server 178 may be configured to retrieve a dialysate prescription of a patient associated with dialysis machine 104 and/or recharger 106 from a memory and communicate the dialysis prescription via communication link 180, 182.

In some examples, processing circuitry 158 is configured to determine a quantity of dialysate chemicals consumed during a dialysis session based on the dialysate prescription and the dialysate amount determined for the dialysis session. For example, processing circuitry 158 may determine a composition of dialysate described by the dialysate prescription. Processing circuitry 158 may use the composition to determine the quantity of dialysate chemicals consumed during a dialysis session by evaluating the amount of dialysate chemicals necessary to substantially maintain (e.g., maintain or nearly maintain to the extent permitted by tolerances of system 100) the composition in an amount of dialysate (e.g., a volume or mass) described by the dialysate amount. In examples, the dialysate prescription describes the composition of the dialysate as an amount and/or concentration of one or more specific constituents (e.g., electrolytes and/or glucose), and the dialysate chemicals include the one or more specific constituents. Processing circuitry 158 may be configured to determine a quantity of the specific constituent consumed during a dialysis treatment based on the amount and/or concentration of the specific constituent described by the dialysate prescription and the amount of the specific constituent necessary to substantially maintain that amount and/or concentration in an amount of dialysate described by the dialysate amount. In examples, dialysis system 100 is configured to generate a solution of the specific constituent using a solid or powder comprising the specific constituent, and processing circuitry 158 is configured to determine a quantity of the solid or powder consumed during the dialysis treatment.

Processing circuitry 158 may be configured to track an inventory of dialysate chemicals based on the determined quantity of dialysate chemicals consumed during one or more dialysis treatments. In examples, processing circuitry 158 is configured to track an inventory of a specific constituent of a dialysate based on the determined quantity of the specific constituent consumed during a dialysis treatment. The inventory may be, for example, a quantity of dialysate chemicals associated with and accessible to a dialysis patient using dialysis system 100, e.g., immediately accessible at the same location as dialysis machine 104 without waiting for a shipment from a consumables supplier.

In examples, processing circuitry 158 is configured to compare the dialysate chemical inventory to a threshold dialysate chemical inventory associated with the patient. Processing circuitry 158 may be configured to issue a communication (e.g., to supply server 178) when the dialysate chemical inventory falls to be at or below or is anticipated to fall below the threshold dialysate chemical inventory for the patient. Supply server 178 may schedule a delivery of the needed dialysate chemicals to the location of the patient based on the communication such that, for example, the patient maintains a sufficient inventory of dialysate chemicals to conduct a prescribed treatment regimen, e.g., for a predetermined duration of time (e.g., a day, several days, a week, several weeks, or more). In some cases, the predetermined duration of time can be based on the proximity of the home of the patient (or other location of dialysis machine 104) to the warehouse or other supplier shipment location.

In examples, supply server 178 schedules a delivery of dialysate chemicals based on a location of the patient, a location of a supply center, an anticipated transit time from the supply center to the patient, and/or other factors impacting the delivery of dialysate chemicals to the patient. Processing circuitry 158 may schedule the delivery of the dialysate chemicals to the location of the patient such that, for example, the patient maintains a sufficient inventory of dialysate chemicals to conduct a prescribed treatment regimen.

In some examples, in addition to or instead of tracking the dialysate consumables, processing circuitry 158 may be configured to determine an expended capacity of a sorbent cartridge (e.g., sorbent cartridge 116, 160) expended during a dialysis treatment. For example, sorbent cartridge 116, 160 may have a defined capacity (e.g., defined by a manufacturers specification, or by some other method) to absorb waste products from dialysate circulating within dialysate circuit 112 during a dialysis treatment. Sorbents within sorbent cartridge 116, 160 may have a defined capacity for sorbing (e.g., absorbing or adsorbing) the waste products. Processing circuitry 158 may be configured to determine an expended capacity of sorbent cartridge 116, 160 based on a portion of the defined capacity expended during a dialysis treatment.

In some examples, the sorbents within sorbent cartridge 116, 160 are configured to remove waste products from the dialysate through an ion exchange. In examples, the ion exchange is enabled by a plurality of ion exchange sites on a sorbent which are accessible to the dialysate as the dialysate flows through sorbent cartridge 116, 160. The waste products of the dialysate may substantially occupy one or more ion exchange sites as the dialysate flows through sorbent cartridge 116, 160, such that the waste products are substantially removed from the dialysate. As the ion exchange sites are occupied, the number of ion exchange sites remaining available to the dialysate decreases, such that some portion of the defined capacity of sorbent cartridge 116, 160 is expended. In examples, sorbent cartridge 116, 160 includes one or more specific sorbents, with a specific sorbent configured to remove a specific waste product from the dialysate, such as ammonium, metal ions, uric acid, creatine, and other uremic toxins. The defined capacity of sorbent cartridge 116, 160 may be based on the individual capacities of the specific sorbents to remove a specific waste product. For example, the defined capacity of sorbent cartridge 116, 160 may be based on expending the individual capacity of a single specific sorbent within the one or more sorbents of sorbent cartridge 116, 160.

In some examples, processing circuitry 158 is configured to determine the expended usable capacity of one or more particular sorbent cartridge 116, 160 based on the dialysate amount determined during a dialysis treatment. For example, processing circuitry 158 is configured to determine the expended capacity of sorbent cartridge 116, 160 using the dialysis amount and a dialysate prescription of the dialysate. Processing circuitry 158 may be configured to treat the dialysate amount as indicative of a quantity (e.g., a volume or mass) of dialysate that sorbent cartridge 116, 160 was exposed to over the dialysis treatment, and determine the expended usable capacity of sorbent cartridge 116, 160 based on the quantity of dialysate. Processing circuitry 158 may be configured substantially treat the dialysate prescription as indicative of a quantity (e.g., a volume or mass) of waste products present in the dialysate, and determine the expended usable capacity of sorbent cartridge 116, 160 based on the quantity of waste products. In examples, sorbent cartridge 116, 160 includes a plurality of modules configured to mechanically connect to define cartridge housing 141 (FIG. 1), and processing circuitry 158 is configured to determine the expended usable capacity of a specific module included in the plurality of modules.

Processing circuitry 158 may be configured to associate the expended usable capacity with sorbent cartridge 116, 160 such that, for example, the expended usable capacity may be determined during a recharge session of sorbent cartridge 116, 160 (e.g., by recharger 106). Processing circuitry 158 may be configured to associate the expended usable capacity with sorbent cartridge 116, 160 using reader 177, 179, which can also be configured as a writer that is configured to write to a memory (e.g., a RFID tag) of the respective cartridge 116 or 160. In other examples, processing circuitry 158 is configured to associate the expended usable capacity with the respective sorbent cartridge 116, 160 by associating the expended usable capacity (or, as a corollary in any of these examples, a remining usable capacity) with the respective identifiers 143, 161. The expended usable capacity may be expressed and/or recorded by processing circuitry 158 using any parameter indicative of a capability of the one or more sorbents in sorbent cartridge 116, 160 to remove waste products from a dialysate, including a remaining portion (e.g., a percentage) of a defined capacity, a portion of the defined capacity expended during a dialysis treatment, a parameter defined by a dialysate amount and/or dialysate prescription of a dialysis treatment, or some other parameter. Processing circuitry 176 of recharger 106 (or another device) may then subsequently determine the expended usable capacity associated with the particular cartridge 116 or 160 based on the capacity determined by processor 158 and associated with the respective cartridge.

Processing circuitry 158 may be configured to substantially monitor and/or update a status of sorbent cartridge 116, 160 based on the expended usable capacity. For example, processing circuitry 158 may designate sorbent cartridge 116, 160 as a used cartridge based on the expended usable capacity. Processing circuitry 158 may be configured to generate a notification and/or prevent a flow of dialysate through dialyzer 110 if the used cartridge is installed within certain portions (e.g., dialysis machine 104) of dialysis system 100. In examples, processing circuitry 158 is configured to alert and/or prevent the flow of dialysate through dialyzer 110 when a used cartridge is installed in dialysate circuit 112.

Processing circuitry 158 may be configured to designate sorbent cartridge 116, 160 as a used cartridge to alert and/or prevent the flow of dialysate through dialyzer 110 based on different or additional criteria. In examples, processing circuitry 158 is configured to identify a single use cartridge used during a dialysis treatment and designate the single use cartridge as a used cartridge. Processing circuitry 158 may be configured to designate sorbent cartridge 116, 160 as a used cartridge based on determining the respective cartridge 116, 160 exceeds a predetermined number of uses over a plurality of dialysis treatments, exceeds a predetermined number of recharges over a plurality of cartridge recharges, following an expiration date associated with the cartridge, or based on some other use limitation. In some examples, processing circuitry 158 is configured to record a time associated with the recharger 106 recharging sorbent cartridge 116, 160 and designate the respective sorbent cartridge 116, 160 as a used cartridge when a predetermined time period has elapsed since the recorded time. The predetermined time periods, predetermined number of uses, predetermined number of recharges, or other parameters used by processing circuitry 158 to define use limitations can be stored by a memory of system 100, e.g., of dialysate machine 104 or recharger 106.

In some examples, processing circuitry 158 is configured to track an inventory of sorbent cartridges based on the designation of a sorbent cartridge as a used cartridge. The inventory may be, for example, a quantity of sorbent cartridges associated with and accessible to a dialysis patient using dialysis system 100. In examples, processing circuitry 158 may compare the sorbent cartridge inventory to a threshold cartridge inventory associated with the patient. Processing circuitry 158 may be configured to issue a communication (e.g., to supply server 178) when the sorbent cartridge inventory falls to be at or below or is anticipated to fall below the threshold cartridge inventory for the patient. Supply server 178 may schedule a delivery of the needed sorbent cartridges to the location of the patient based on the communication. In examples, supply server 178 schedules a delivery of sorbent cartridges based on a location of the patient, a location of a supply center, an anticipated transit time from the supply center to the patient, and/or other factors impacting the delivery of sorbent cartridges to the patient.

As discussed, dialysis system 100 (e.g., recharger 106) may be configured to recharge sorbent cartridge 116, 160 following a dialysis treatment to substantially restore the capacity of sorbent cartridge 116, 160. Recharger 106 is configured to recharge sorbent cartridge 116, 160 to substantially restore its usable capacity using one or more recharge chemicals. Recharger 106 may be configured to expose the one or more sorbents of sorbent cartridge 116, 160 to the recharge chemicals to substantially restore the usable capacity. In examples, recharger 106 includes container 117, 119, 121 configured to hold one or more solutions of the recharge chemicals. Recharger 106 may be configured to flow the one or more solutions through sorbent cartridge 116, 160 to substantially restore its usable capacity.

The recharge chemicals may be configured to recharge the one or more sorbents within sorbent cartridge 116, 160 through an ion exchange. The recharge chemicals may be configured to exchange ions with one or more sorbents at ion exchange sites substantially occupied by the waste products removed during a prior dialysis treatment. The recharge chemicals (e.g., in the form of a solution) may substantially replace a waste product occupying an ion exchange site, such that the waste product is substantially removed from the sorbent. As the recharge chemicals remove waste products from the ion exchange sites of the sorbent, the capacity of the sorbent for waste removal may be substantially restored. Recharger 106 may be configured to substantially flush recharge chemicals held within container 117, 119, 121 through sorbent cartridge 116, 160 to remove waste products and restore the capacity. In examples, sorbent cartridge 116, 160 includes one or more specific sorbents, and recharger 106 is configured to recharge a specific sorbent using a specific recharge chemical. The recharge chemicals applied to sorbent cartridge 116, 160 may be based on the specific sorbents comprising sorbent cartridge 116, 160.

Processing circuitry 158 (or other processing circuitry, such as processing circuitry 176 of recharger 106) may be configured to determine a quantity of recharge chemicals consumed or anticipated to be consumed during a recharging session of sorbent cartridge 116, 160 in order to facilitate automatic reordering of the recharge chemicals or generation of a notification to a user to order more recharge chemicals. In examples, processing circuitry 158 determines the quantity of recharge chemicals based on a dialysate amount determined during a dialysis treatment that used sorbent cartridge 116, 160. In examples, processing circuitry 158 is configured to determine the quantity of recharge chemicals consumed or anticipated to be consumed during a recharging session of sorbent cartridge 116, 160 using the dialysis amount associated with sorbent cartridge 116, 160 and a dialysate prescription of the dialysate. In some examples, processing circuitry 158 is configured to determine the quantity of recharge chemicals consumed or anticipated to be consumed during a recharging session of sorbent cartridge 116, 160 using an expended usable capacity associated with sorbent cartridge 116, 160, where the expended usable capacity is based on a dialysate amount and/dialysate prescription.

n some examples, processing circuitry 158 is configured to identify sorbent cartridge 116, 160 as a used cartridge having some expended usable capacity, and determine a quantity of recharge chemicals consumed or anticipated to be consumed based substantially on the status of on sorbent cartridge 116, 160 as a used cartridge. For example, sorbent cartridge 116, 160 may be configured to be recharged using a predetermined and set (e.g., fixed) amount of recharging chemicals substantially regardless of a dialysate amount or dialysate prescription determined during a dialysis treatment. Processing circuitry 158 may be configured to identify sorbent cartridge 116, 160 as a cartridge configured for the set amount of recharging chemicals, and determine the quantity of recharge chemicals based on the set amount.

In examples, recharger 106 is configured to recharge sorbent cartridge 116, 160 using a recharge solution having a specific composition of the recharge chemicals. Processing circuitry 158 may be configured to use the specific composition to determine the quantity of recharge chemicals consumed or anticipated to be consumed during a recharging session. In examples, the specific composition describes an amount and/or concentration of one or more specific recharge constituents, and the recharge chemicals include the one or more specific recharge constituents. Processing circuitry 158 may be configured to determine a quantity of a specific recharge constituent consumed or anticipated to be consumed during a recharge session based on the amount and/or concentration of the specific recharge constituent described by specific composition. In examples, dialysis system 100 is configured to generate a solution of the specific recharge constituent using a solid or powder comprising the specific recharge constituent, and processing circuitry 158 is configured to determine a quantity of the solid or powder consumed or anticipated to be consumed during the recharge session.

In examples, recharger 106 is configured to recharge sorbent cartridge 116, 160 using the quantity of recharge chemicals determined by processing circuitry 158. Processing circuitry 158 may be configured to identify sorbent cartridge 116, 160 using the respective identifiers 143, 161 and the reader 177 or 179 and retrieve or determine the quantity of recharge chemicals anticipated to be consumed in the recharging of sorbent cartridge 116, 160. Recharger 106 may be configured to flush an amount (e.g., a mass or volume) of the recharge chemicals held in container 117, 119, 121 through sorbent cartridge 116, 160 based on the quantity of recharge chemicals determined by processing circuitry 158.

Processing circuitry 158 can determine quantity of recharge chemicals consumed or anticipated to be consumed during a recharging session using any one or more of the techniques described above. Processing circuitry 158 may be configured to track an inventory of recharge chemicals based on the determined quantity of recharge chemicals consumed or anticipated to be consumed during a recharging session. In examples, processing circuitry 158 is configured to track an inventory of a specific recharge constituent based on the determined quantity of the specific recharge constituent consumed or anticipated to be consumed during a recharge session. The inventory may be, for example, a quantity of recharge chemicals associated with and accessible to a dialysis patient using dialysis system 100. In examples, processing circuitry 158 is configured to compare the recharge chemical inventory to a threshold recharge chemical inventory associated with the patient.

Processing circuitry 158 may be configured to facilitate the ordering of the recharge chemicals in response to determining the recharge chemical inventory is less than or equal to the threshold. For example, processing circuitry 158 may issue a communication (e.g., to supply server 178) when the recharge chemical inventory falls to be at or below or is anticipated to fall below the threshold recharge chemical inventory for the patient. Supply server 178 may schedule a delivery of the needed recharge chemicals to the location of the patient based on the communication. In examples, supply server 178 schedules a delivery of recharge chemicals based on a location of the patient, a location of a supply center, an anticipated transit time from the supply center to the patient, and/or other factors impacting the delivery of research chemicals to the patient.

In any of the examples described herein, the ordering of dialysis consumables can be automatic, e.g., with no intervention from the patient or patient caretaker, or can require patient input, e.g., approval of an order by the patient or patient caretaker, before the shipment of dialysis consumables takes place.

In examples, processing circuitry 158 is configured to determine a consumption trend for a patient based on the determined quantity of materials consumed during one or more dialysis treatments. Processing circuitry 158 may be configured to determine a future consumables inventory based on the consumption trend determined, wherein the future consumables inventory is an inventory of consumables estimated to occur at a future point in time. As an example, processing circuitry 158 can be configured to determine the future consumables inventory for a patient based on the inventory of consumables tracked for the patient. Processing circuitry 158 may be configured to evaluate a plurality of dialysis treatments and determine a quantity of materials consumed for each dialysis treatment. Processing circuitry 158 may be configured to determine the consumption trend based on the resulting plurality of quantities of materials consumed. For example, processing circuitry 158 may evaluate the resulting plurality of quantities of materials consumed and determine that the inventory of a specific consumable is decreasing by a certain quantity per unit time. Processing circuitry 158 may substantially extrapolate this consumption trend (e.g., the quantity per unit time) to determine a future point in time when the inventory of the specific consumable for the patient is likely to fall to or below a threshold inventory for the specific consumable. Supply server 178 may schedule a delivery of the needed specific consumable to the location of the patient based on the determined future point in time.

In examples, the quantity of materials consumed includes additional one-time and multi-use items that may be used during the dialysis treatment. For example, the additional items may include dialyzer 110, a tubing set configured to establish blood flow between arterial line 122 and shunt 120 or between venous line 124 and shunt 128, and/or other items consumed during a dialysis treatment. Processing circuitry 158 may be configured to track an inventory of the items. In examples, processing circuitry 158 is configured to compare the inventory of items to a threshold item inventory associated with the patient. Processing circuitry 158 is configured to facilitate the reordering of these consumables. For example, processing circuitry 158 may be configured to issue a communication (e.g., to supply server 178) when the inventory of items falls to be at or below or is anticipated to fall below the threshold item inventory for the patient. Supply server 178 may schedule a delivery of the needed items to the location of the patient based on the communication. In examples, supply server 178 schedules a delivery of the needed items based on a location of the patient, a location of a supply center, an anticipated transit time from the supply center to the patient, and/or other factors impacting the delivery of the needed items to the patient.

FIG. 3 is a block diagram illustrating an example system 200 including processing circuitry 158 of dialysis machine 104, processing circuitry 176 of recharger 106, and processing circuitry 183 of supply server 178. System 200 may be an example of dialysis system 100. System 200 includes an access point 190, a network 192, and one or more other computing devices 196A-196N. Processing circuitry of dialysis machine 104 and/or recharger 106 may be communicatively coupled to network 192 (e.g., via access point 190) in accordance with one or more techniques described herein. For example, dialysis machine 104 and/or recharger 106 may use communication circuitry to communicate with access point 190 via a hard-line or wireless connection. In the example of FIG. 3, processing circuitry 158, processing circuitry 176, supply server 178 (e.g., processing circuitry 183), access point 190, and/or computing devices 196A-196N are interconnected and may communicate with each other through network 192. Access point 190 may include a device that connects to network 192 via any of a variety of connections, such as telephone dial-up, digital subscriber line (DSL), or cable modem connections. In other examples, access point 190 may be coupled to network 192 through different forms of connections, including wired or wireless connections.

Supply server 178 may be configured to provide a secure storage site for data that has been collected from processing circuitry 158, processing circuitry 176, and/or processing circuitry 183. In some cases, supply server 178 may assemble data in web pages or other documents for viewing by trained professionals, such as clinicians, via computing devices 196A-196N. In examples, supply server 178 may comprise one or more servers, a cloud, one or more databases, and/or a data center. Supply server 178 may include a storage device 194 (e.g., a memory device) to, for example, store data retrieved from processing circuitry 158, processing circuitry 176, and/or processing circuitry 183. Processing circuitry 158, processing circuitry 176, and/or processing circuitry 183 may include one or more processors that are configured to implement functionality and/or process instructions for execution within supply server 178. For example, processing circuitry 158, processing circuitry 176, and/or processing circuitry 183 may be capable of processing instructions stored in storage device 194. Processing circuitry 158, processing circuitry 176, and/or processing circuitry 183 may each include, for example, one or more microprocessors, digital signal processors (DSPs), application specific integrated circuits (ASICs), field programmable gate arrays (FPGAs), programmable logic circuitry, or the like, either alone or in any suitable combination. In general, processing circuitry or control circuitry described herein may comprise any suitable arrangement of hardware (e.g., circuitry), alone or in combination with software and/or firmware, to perform the various techniques described herein and attributed to the processing circuitry.

Storage device 194 may include a computer-readable storage medium or computer-readable storage device. In some examples, storage device 194 includes one or more of a short-term memory or a long-term memory, such as random access memory (RAM), read only memory (ROM), non-volatile RAM (NVRAM), programmable read only memory (PROM), erasable programmable read only memory (EPROM), electronically erasable programmable read only memory (EEPROM), magnetic discs, optical discs, flash memories, or forms of EPROM or EEPROM.

In examples, any of processing circuitry 158, processing circuitry 176, and/or processing circuitry 183 may be configured to perform all or some portion of the functionality described individually with respect to processing circuitry 158, processing circuitry 176, or processing circuitry 183. Dialysis machine 104 and/or recharger 106 may be configured to provide data to supply server 178 to enable processing circuitry 183 to perform any portion of the functionality described with respect to processing circuitry 158 and/or processing circuitry 176. For example, processing circuitry 158, processing circuitry 176, or processing circuitry 183 may be configured to determine and communicate a dialysate amount associated with a dialysis treatment delivered by dialysis machine 104 based on one or more communications from or within dialysis machine 104 and/or recharger 106. Processing circuitry 158, processing circuitry 176, or processing circuitry 183 may be configured to determine and communicate a dialysate prescription associated with the dialysis treatment based on one or more communications from dialysis machine 104, recharger 106, and/or supply server 178. Processing circuitry 158, processing circuitry 176, or processing circuitry 183 may be configured to determine and communicate a quantity of materials consumed based on one or more communications from dialysis machine 104, recharger 106, and/or supply server 178. Processing circuitry 158, processing circuitry 176, or processing circuitry 183 may be configured to determine and communicate a consumption trend based on one or more communications from dialysis machine 104, recharger 106, and/or supply server 178. Processing circuitry 158, processing circuitry 176, or processing circuitry 183 may be configured to determine an alert threshold and communicate an alert based on one or more communications from dialysis machine 104, recharger 106, and/or supply server 178. Processing circuitry 158, processing circuitry 176, or processing circuitry 183 may be configured to schedule a delivery of consumables to the location of a patient based on one or more communications from dialysis machine 104, recharger 106, and/or supply server 178. In some examples, processing circuitry 158, 176 comprises first processing circuitry, and processing circuitry 183 comprises second processing circuitry. Processing circuitry 158, 176 may be configured to issue a communication to the processing circuitry 183, where the communication is indicative of a quantity of materials consumed.

A technique for determining a quantity of materials consumed during at least one of a dialysis machine delivering the dialysis treatment or a recharger recharging a sorbent cartridge is illustrated in FIG. 4. Although the technique is described mainly with reference to dialysis system 100 and system 200 of FIGS. 1-3, the technique may be applied to other dialysis systems in other examples.

The technique includes determining, by processing circuitry 158 of dialysis machine 104, processing circuitry 176 of recharger 106, and/or processing circuitry 183 of supply server 178, a dialysate prescription and a dialysate amount (402). The dialysate prescription is indicative of a composition of a dialysate using during a dialysis treatment. The dialysate amount is indicative of an amount of dialysate transferred through dialyzer 110 during the dialysis treatment. Dialysis machine 104 may be configured to deliver a dialysis treatment to a patient by transferring the dialysate through dialyzer 110 and recycling the dialysate using sorbent cartridge 116, 160. Recharger 106 may be configured to recharge sorbent cartridge 116, 160 using one or more recharge chemicals.

Processing circuitry 158 is primarily referred to throughout the remainder of the description of FIG. 4 for ease of description, but all or part of the technique of FIG. 4 may be performed by processing circuitry 176 and/or 183 or other processing circuitry alone or in combination with processing circuitry 158 in other examples.

Processing circuitry 158 may determine the dialysate prescription using any suitable technique, such as by receiving the prescription via input device 186, via a remote device, such as a clinician computing device or supply server 178. Supply server 178 may, for example, retrieve a dialysate prescription of a patient associated with dialysis machine 104 and/or recharger 106 from a memory and communicate the dialysis prescription via communication link 180, 182.

As discussed above, in some examples, processing circuitry 158 may determine the dialysate amount based on a flow rate of dialysate within dialysate circuit 112 of dialysis machine 104. In examples, processing circuitry 158 determines the flow rate on a pump capacity of dialysate pump 118. Processing circuitry 158 may determine the dialysate amount based on an operating time of dialysate pump 118 and the pump capacity during the operating time. In examples, processing circuitry 158 determines the flow rate based on a direct measurement of the flow rate using, for example, flow sensor 184. Processing circuitry 158 may be configured to determine the dialysate amount based on the determined flow rate and a time over which the flow rate substantially occurred. Processing circuitry 158 may determine the time over which the flow rate occurred using a communication from dialysate pump 118, a communication from flow sensor 184, and/or a communication from another component of dialysis system 100 and/or system 200.

Processing circuitry 158 determines a quantity of materials consumed during a dialysis treatment using the dialysate prescription and the dialysate amount (404). In some examples, the materials include dialysate chemicals consumed during the dialysis treatment and processing circuitry 158 determines the amount of dialysate chemicals necessary to substantially maintain a composition of the dialysate in an amount of dialysate described by the dialysate amount. In examples, the dialysate prescription describes the composition of the dialysate as an amount and/or concentration of one or more specific constituents, and processing circuitry 158 determines a quantity of the specific constituent consumed during the dialysis treatment. In examples, processing circuitry 158 determines a quantity of a solid or powder consumed to generate a solution of one or more of the dialysate chemicals.

In addition to or instead of the dialysate chemicals, in some examples, the materials consumed during a dialysis treatment relates to cartridges 116, 160. For example, processing circuitry 158 may determine an expended capacity of one or more sorbent cartridges 116, 160 expended during the dialysis treatment. For example, sorbent cartridge 116, 160 may have a defined capacity (e.g., defined by a manufacturers specification, or by some other method) to absorb waste products from dialysate circulating within dialysate circuit 112 during a dialysis treatment. The defined capacity of sorbent cartridge 116, 160 may be based on the individual capacities of the specific sorbents to remove one or more specific waste products. Processing circuitry 158 determine the expended usable capacity of sorbent cartridge 116, 160 based on the dialysate amount and the dialysate prescription. In examples, processing circuitry 158 determines an expended usable capacity of one or more specific modules of sorbent cartridge 116, 160, The one or more specific modules may mechanically connect to define cartridge housing 141 of sorbent cartridge 116.

Processing circuitry 158 may associate the expended usable capacity with sorbent cartridge 116, 160 using any suitable technique, such as via storing the cartridge identifier (e.g., as indicated by indicators 143, 161) with the determined in expended usable capacity memory of system 100 or by writing the expended usable capacity to the respective indicator 143, 161. Processing circuitry 158 may substantially monitor and/or update a status of sorbent cartridge 116, 160 based on the expended usable capacity. In examples, processing circuitry 158 designates sorbent cartridge 116, 160 as a used cartridge based on the expended usable capacity. Processing circuitry 158 may alert and/or prevent a flow of dialysate through dialyzer 110 if the used cartridge is installed in dialysate circuit 112 of dialysis machine 104. In examples, processing circuitry 158 designates sorbent cartridge 116, 160 as a used cartridge based on a single use of sorbent cartridge 116, 160, exceeding a limited number of uses over a plurality of dialysis treatments, exceeding a limited number of recharges over a plurality of recharge session, following an expiration date associated with cartridge 116, 160, or based on some other use limitation.

Another example of materials related to cartridges 116, 160 includes recharge chemicals. Processing circuitry 158 may, for example, determine a quantity of recharge chemicals consumed or anticipated to be consumed during a recharging session of sorbent cartridge 116, 160. In examples, processing circuitry 158 may determine the quantity of recharge chemicals based on a dialysate amount determined during a dialysis treatment that used sorbent cartridge 116, 160. In examples, processing circuitry 158 determines the quantity of recharge chemicals using the dialysate prescription. In examples, processing circuitry 158 determines the quantity of recharge chemicals using the expended usable capacity associated with sorbent cartridge 116, 160. In some examples, sorbent cartridge 116, 160 is configured to be recharged using a set amount of recharging chemicals, and processing circuitry 158 determines the quantity of recharge chemicals based on the set amount. In examples, recharger 106 recharges sorbent cartridge 116, 160 using a recharge solution having a specific composition of the recharge chemicals, and processing circuitry 158 uses the specific composition to determine the quantity of recharge chemicals consumed or anticipated to be consumed. In examples, processing circuitry 158 determines a quantity of a solid or powder consumed to generate a solution of one or more of the recharge chemicals.

Processing circuitry 158 may be configured to track an inventory of consumables based on the quantity of consumables consumed during the dialysis treatment. Processing circuitry 158 may be configured to track an inventory of dialysate chemicals based on the determined quantity of dialysate chemicals consumed during a dialysis treatment. Processing circuitry 158 may be configured to track an inventory of recharge chemicals based on the determined quantity of recharge chemicals consumed or anticipated to be consumed during a recharge session. Processing circuitry 158 may be configured to track an inventory of sorbent cartridges based on the designation of a sorbent cartridge as a used cartridge. The inventory may be, for example, a quantity of consumables associated with and accessible to a dialysis patient using dialysis system 100. In examples, processing circuitry 158 compares the inventory of consumables threshold consumables inventory associated with the patient. Processing circuitry 158 may issue a communication (e.g., to supply server 178) when the inventory of consumables falls to be at or below or is anticipated to fall below a threshold consumables inventory for the patient. Supply server 178 may schedule a delivery of the needed dialysate chemicals to the location of the patient based on the communication. In examples, supply server 178 schedules the delivery of consumables based on a location of the patient, a location of a supply center, an anticipated transit time from the supply center to the patient, and/or other factors impacting the delivery of consumables to the patient.

In examples, processing circuitry 158 determines a consumption trend for a patient based on the determined quantity of materials consumed. Processing circuitry 158 may determine a future consumables inventory based on the consumption trend determined. In examples, processing circuitry 158 evaluates a plurality of dialysis treatments and determines a quantity of materials consumed for each dialysis treatment to determine the consumption trend. Processing circuitry 158 may substantially extrapolate the consumption trend to determine a future point in time when the inventory of consumables is likely to fall to or below the threshold consumables inventory. Supply server 178 may schedule a delivery of consumables to the patient based on the determined future point in time.

The techniques described in this disclosure, including those attributed to processing circuitry 158, 176, 183, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry. The term “processor,” “controller,” “processing circuitry,” or “control circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

The present disclosure includes the following examples.

Example 1: A dialysis system includes a dialysis machine configured to deliver a dialysis treatment to a patient by at least transferring a dialysate through a dialyzer, wherein the dialysis machine is configured to recycle the dialysate using a cartridge; a recharger configured to recharge the cartridge following the dialysis treatment; and processing circuitry configured to: determine a dialysate prescription indicative of a composition of the dialysate, determine a dialysate amount indicative of an amount of dialysate transferred through the dialyzer during the dialysis treatment, and determine, using the dialysate amount and the dialysate prescription, a quantity of materials consumed during at least one of the dialysis machine delivering the dialysis treatment or the recharger recharging the cartridge.

Example 2: The dialysis system of example 1, wherein the processing circuitry is configured to track an inventory of consumables associated with the patient based on the quantity of materials consumed.

Example 3: The dialysis system of example 2, wherein the processing circuitry is configured to: determine the inventory of consumables is below or is anticipated to fall below a threshold consumables inventory associated with the patient, wherein the threshold consumables inventory is based on the dialysate prescription, and issue a communication in response to determining the inventory of consumables is below or is anticipated to fall below the threshold consumables inventory.

Example 4: The dialysis system of any of examples 1-3, wherein the dialysis machine includes a dialysis pump configured to transfer the dialysate through the dialyzer, and wherein the dialysate amount is based on a flow rate of the dialysate provided by the dialysate pump.

Example 5: The dialysis system of any of examples 1-4, wherein the processing circuitry is configured to communicate the quantity of materials consumed to a remote device.

Example 6: The dialysis system of any of examples 1-5, wherein the recharger is configured to recharge the cartridge using a quantity of recharge chemicals, and wherein the processing circuitry is configured to determine the quantity of recharge chemicals required to recharge the cartridge based on the dialysate amount and the dialysate prescription.

Example 7: The dialysis system of example 6, wherein the processing circuitry is configured to determine the quantity of recharge chemicals by at least identifying a cartridge identifier associated with the cartridge.

Example 8: The dialysis system of any of examples 1-7, wherein the quantity of materials consumed includes a quantity of cartridges consumed in connection with the dialysis treatment.

Example 9: The dialysis system of any of examples 1-8, wherein the processing circuitry is configured to: record a time associated with the recharger recharging the cartridge, and include the cartridge in the quantity of materials consumed when a predetermined time period has elapsed since the recorded time.

Example 10: The dialysis system of any of examples 1-9, wherein the dialysis machine is configured to add dialysate chemicals to the dialysate during the dialysis treatment, and wherein the processing circuitry is configured to determine a quantity of dialysate chemicals consumed during the dialysis treatment based on the dialysate amount and the dialysate prescription.

Example 11: The dialysis system of any of examples 1-10, wherein the processing circuitry is configured to: determine a consumption trend for the patient based on quantities of materials consumed during at least one of a plurality of dialysis treatments or a plurality of cartridge recharges, and determine, based on the consumption trend, a future consumables inventory, wherein the future consumables inventory is an estimation of the consumables inventory at a future point in time.

Example 12: The dialysis system of any of examples 1-11, wherein: the dialysis system includes a remote device, the processing circuitry includes first processing circuitry and second processing circuitry, the dialysis machine, the recharger, or the dialysis machine and the recharger include the first processing circuitry, the remote device includes the second processing circuitry, and the first processing circuitry is configured to issue a communication to the second processing circuitry, wherein the communication is indicative of the quantity of materials consumed.

Example 13: The dialysis system of any of examples 1-12, wherein: the inventory of consumables includes an individual inventory for each consumable of a plurality of individual consumables, and the processing circuitry is configured to determine the quantity of materials consumed by at least determining a quantity of each individual consumable of the plurality of individual consumables consumed.

Example 14: The dialysis system of any of examples 1-13, wherein: the dialysis machine, the recharger, or the dialysis machine and the recharger are located at a first location; and the processing circuitry is configured to schedule a delivery of one or more consumables to the first location from a second location geographically displaced from the first location based on the quantity of materials consumed.

Example 15: The dialysis system of any of examples 1-14, wherein the quantity of materials consumed includes at least one of a quantity of dialyzers consumed during the dialysis treatment or a quantity of one or more tubing sets consumed during the dialysis treatment.

Example 16: A dialysis system includes a dialysis machine configured to deliver a dialysis treatment to a patient by at least transferring a dialysate through a dialyzer, and wherein the dialysis machine is configured to recycle the dialysate using a cartridge; a recharger configured to recharge the cartridge following the dialysis treatment, wherein the dialysis machine, the recharger, or the dialysis machine and the recharger are located at a first location; and processing circuitry configured to: determine a dialysate prescription indicative of a composition of the dialysate, determine a dialysate amount indicative of an amount of dialysate transferred through the dialyzer during the dialysis treatment, determine a quantity of materials consumed during at least one the dialysis machine delivering the dialysis treatment or the recharger recharging the cartridge based on the dialysate amount and the dialysate prescription, track an inventory of consumables associated with the patient based on the quantity of materials consumed, compare the inventory of consumables with a threshold consumables inventory associated with the patient, wherein the threshold consumables inventory is based on the dialysate prescription, and schedule a delivery of one or more consumables to the first location from a second location geographically displaced from the first location based on the consumables inventory tracked.

Example 17: The dialysis system of example 16, wherein the quantity of materials consumed includes a quantity of recharge chemicals required to recharge the cartridge, and wherein the processing circuitry is configured to: determine the quantity of recharge chemicals required based on the dialysate amount and the dialysate prescription, and track an inventory of recharge chemicals associated with the patient based on the quantity of recharge chemicals required to recharge the cartridge.

Example 18: The dialysis system of example 16 or example 17, wherein the quantity of materials consumed includes a quantity of dialysate chemicals added to the dialysate, and wherein the processing circuitry is configured to: determine the quantity of dialysate chemicals required based on the dialysate amount and the dialysate prescription, and track an inventory of dialysate chemicals associated with the patient based on the quantity of dialysate chemicals added to the dialysate.

Example 19: A method includes determining, by processing circuitry, a dialysate prescription indicative of a composition of a dialysate, wherein a dialysis system includes a dialysis machine and a recharger, wherein the dialysis machine is configured to deliver a dialysis treatment to a patient by at least transferring the dialysate through a dialyzer and recycling the dialysate using a cartridge; determining, by the processing circuitry, a dialysate amount indicative of an amount of dialysate transferred through the dialyzer during the dialysis treatment, and wherein the recharger is configured to recharge the cartridge; and determining, by the processing circuitry, a quantity of materials consumed during at least one of the dialysis machine delivering the dialysis treatment or the recharger recharging the cartridge, wherein the system circuitry determines the quantity of material consumed based on the dialysate amount and the dialysate prescription.

Example 20: The method of example 19, further includes determining, by the processing circuitry, an inventory of consumables associated with the patient based on the quantity of materials consumed; and determining, by the processing circuitry, the inventory of consumables is below or anticipated to fall below a threshold consumables inventory associated with the patient, wherein the threshold consumables inventory is based on the dialysate prescription; and issuing a communication in response to determining the inventory of consumables is below or anticipated to fall below the threshold consumables inventory.

Various examples of the disclosure have been described. Any combination of the described systems, operations, or functions is contemplated. These and other examples are within the scope of the following claims.

What is claimed is:

1. A dialysis system comprising:
a dialysis machine configured to deliver a dialysis treatment to a patient by at least transferring a dialysate through a dialyzer, wherein the dialysis machine is configured to recycle the dialysate using a cartridge;
a recharger configured to recharge the cartridge following the dialysis treatment; and
processing circuitry configured to:
determine a dialysate prescription indicative of a composition of the dialysate,
determine a dialysate amount indicative of an amount of dialysate transferred through the dialyzer during the dialysis treatment, and
determine, using the dialysate amount and the dialysate prescription, a quantity of materials consumed during at least one of the dialysis machine delivering the dialysis treatment or the recharger recharging the cartridge.

2. The dialysis system of claim 1, wherein the processing circuitry is configured to track an inventory of consumables associated with the patient based on the quantity of materials consumed.

3. The dialysis system of claim 2, wherein the processing circuitry is configured to:
determine the inventory of consumables is below or is anticipated to fall below a threshold consumables inventory associated with the patient, wherein the threshold consumables inventory is based on the dialysate prescription, and
issue a communication in response to determining the inventory of consumables is below or is anticipated to fall below the threshold consumables inventory.

4. The dialysis system of claim 1, wherein the dialysis machine includes a dialysis pump configured to transfer the dialysate through the dialyzer, and wherein the dialysate amount is based on a flow rate of the dialysate provided by the dialysate pump.

5. The dialysis system of claim 1, wherein the processing circuitry is configured to communicate the quantity of materials consumed to a remote device.

6. The dialysis system of claim 1, wherein the recharger is configured to recharge the cartridge using a quantity of recharge chemicals, and wherein the processing circuitry is configured to determine the quantity of recharge chemicals required to recharge the cartridge based on the dialysate amount and the dialysate prescription.

7. The dialysis system of claim 6, wherein the processing circuitry is configured to determine the quantity of recharge chemicals by at least identifying a cartridge identifier associated with the cartridge.

8. The dialysis system of claim 1, wherein the quantity of materials consumed includes a quantity of cartridges consumed in connection with the dialysis treatment.

9. The dialysis system of claim 1, wherein the processing circuitry is configured to:
record a time associated with the recharger recharging the cartridge, and
include the cartridge in the quantity of materials consumed when a predetermined time period has elapsed since the recorded time.

10. The dialysis system of claim 1, wherein the dialysis machine is configured to add dialysate chemicals to the dialysate during the dialysis treatment, and wherein the processing circuitry is configured to determine a quantity of dialysate chemicals consumed during the dialysis treatment based on the dialysate amount and the dialysate prescription.

11. The dialysis system of claim 1, wherein the processing circuitry is configured to:
determine a consumption trend for the patient based on quantities of materials consumed during at least one of a plurality of dialysis treatments or a plurality of cartridge recharges, and
determine, based on the consumption trend, a future consumables inventory, wherein the future consumables inventory is an estimation of the consumables inventory at a future point in time.

12. The dialysis system of claim 1, wherein:
the dialysis system includes a remote device,
the processing circuitry includes first processing circuitry and second processing circuitry,
the dialysis machine, the recharger, or the dialysis machine and the recharger include the first processing circuitry,
the remote device includes the second processing circuitry, and
the first processing circuitry is configured to issue a communication to the second processing circuitry, wherein the communication is indicative of the quantity of materials consumed.

13. The dialysis system of claim 1, wherein:
the inventory of consumables includes an individual inventory for each consumable of a plurality of individual consumables, and
the processing circuitry is configured to determine the quantity of materials consumed by at least determining a quantity of each individual consumable of the plurality of individual consumables consumed.

14. The dialysis system of claim 1, wherein:
the dialysis machine, the recharger, or the dialysis machine and the recharger are located at a first location; and
the processing circuitry is configured to schedule a delivery of one or more consumables to the first location from a second location geographically displaced from the first location based on the quantity of materials consumed.

15. The dialysis system of claim 1, wherein the quantity of materials consumed includes at least one of a quantity of dialyzers consumed during the dialysis treatment or a quantity of one or more tubing sets consumed during the dialysis treatment.

16. A dialysis system comprising:
a dialysis machine configured to deliver a dialysis treatment to a patient by at least transferring a dialysate through a dialyzer, and wherein the dialysis machine is configured to recycle the dialysate using a cartridge;
a recharger configured to recharge the cartridge following the dialysis treatment, wherein the dialysis machine, the recharger, or the dialysis machine and the recharger are located at a first location; and
processing circuitry configured to:
determine a dialysate prescription indicative of a composition of the dialysate,
determine a dialysate amount indicative of an amount of dialysate transferred through the dialyzer during the dialysis treatment,
determine a quantity of materials consumed during at least one the dialysis machine delivering the dialysis treatment or the recharger recharging the cartridge based on the dialysate amount and the dialysate prescription,
track an inventory of consumables associated with the patient based on the quantity of materials consumed,
compare the inventory of consumables with a threshold consumables inventory associated with the patient, wherein the threshold consumables inventory is based on the dialysate prescription, and
schedule a delivery of one or more consumables to the first location from a second location geographically displaced from the first location based on the consumables inventory tracked.

17. The dialysis system of claim 16, wherein the quantity of materials consumed includes a quantity of recharge chemicals required to recharge the cartridge, and wherein the processing circuitry is configured to:
determine the quantity of recharge chemicals required based on the dialysate amount and the dialysate prescription, and
track an inventory of recharge chemicals associated with the patient based on the quantity of recharge chemicals required to recharge the cartridge.

18. The dialysis system of claim 16, wherein the quantity of materials consumed includes a quantity of dialysate chemicals added to the dialysate, and wherein the processing circuitry is configured to:
determine the quantity of dialysate chemicals required based on the dialysate amount and the dialysate prescription, and
track an inventory of dialysate chemicals associated with the patient based on the quantity of dialysate chemicals added to the dialysate.

19. A method, comprising:
determining, by processing circuitry, a dialysate prescription indicative of a composition of a dialysate, wherein a dialysis system includes a dialysis machine and a recharger, wherein the dialysis machine is configured to deliver a dialysis treatment to a patient by at least transferring the dialysate through a dialyzer and recycling the dialysate using a cartridge;
determining, by the processing circuitry, a dialysate amount indicative of an amount of dialysate transferred through the dialyzer during the dialysis treatment, and wherein the recharger is configured to recharge the cartridge; and
determining, by the processing circuitry, a quantity of materials consumed during at least one of the dialysis machine delivering the dialysis treatment or the recharger recharging the cartridge, wherein the system circuitry determines the quantity of material consumed based on the dialysate amount and the dialysate prescription.

20. The method of claim 19, further comprising:
determining, by the processing circuitry, an inventory of consumables associated with the patient based on the quantity of materials consumed; and
determining, by the processing circuitry, the inventory of consumables is below or anticipated to fall below a threshold consumables inventory associated with the patient, wherein the threshold consumables inventory is based on the dialysate prescription; and
issuing a communication in response to determining the inventory of consumables is below or anticipated to fall below the threshold consumables inventory.
